SEQUENCE LISTING (13 sequences)

SEQ ID NO: 1 - RNA, 19 nt, Homo sapiens
guauaccaggucuccucaa

SEQ ID NO: 2 - RNA, 19 nt, Homo sapiens
cagucgcguuugcgacugg

SEQ ID NO: 3 - RNA, 19 nt, Homo sapiens
gaaucaauaugucacucua

SEQ ID NO: 4 - RNA, 19 nt, Homo sapiens
acucaaguccagccauaag

SEQ ID NO: 5 - protein, 15 aa, Homo sapiens
CTEHVRAPPAPSRSR

SEQ ID NO: 6 - protein, 17 aa, Homo sapiens
MHSIRTVEIKVPEIEEC

SEQ ID NO: 7 - protein, 16 aa, Homo sapiens
KDSQALQTGELDSAHS

SEQ ID NO: 8 - protein, 15 aa, Homo sapiens
CRQKKGKSLYKIKPR

SEQ ID NO: 9 - protein, 17 aa, Homo sapiens
CKPRHDSGIKAKISMKT

SEQ ID NO: 10 - DNA, 4349 bp, Homo sapiens
atgcattcgattcgcacggtggagattaaagtccccgagatagaggaaacgtttttcgcgcccaggttcagcgaggagccgcgcgggggcagagggggcggcggcggcgggcggggagccaggcccgagctgcgttctgcgcagccattggtgggcgccgcgctctgcactgagcatgttcgcgccccgccggcccctagccgcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaag

SEQ ID NO: 11 - DNA, 588 bp, Homo sapiens
gaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacc

SEQ ID NO: 12 - DNA, 6633 bp, Homo sapiens
atgcattcgattcgcacggtggagattaaagtccccgagatagaggaaacgtttttcgcgcccaggttcagcgaggagccgcgcgggggcagagggggcggcggcggcgggcggggagccaggcccgagctgcgttctgcgcagccattggtgggcgccgcgctctgcactgagcatgttcgcgccccgccggcccctagccgcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaaga

SEQ ID NO: 13 - protein, 1865 aa, Homo sapiens
MHSIRTVEIKVPEIEETFFAPRFSEEPRGGRGGGGGGRGARPELRSAQPLVGAALCTEHVRAPPAPSRSRSRSDGSHGSRRALCKAAADASGASRARVGLPSQRLPPPPPDSPGRLPRPGPGAEEGRDGLSARGAAAQQRDCTMGNQDGKLKRSAGDALHEGGGGAEDALGPRDVEATKKGSGGKKALGKHGKGGGGGGGGGESGKKKSKSDSRASVFSNLRIRKNLSKGKGAGGSREDVLDSQALQTGELDSAHSLLTKTPDLSLSADEAGLSDTECADPFEVTGPGGPGPAEARVGGRPIAEDVETAAGAQDGQRTSSGSDTDIYSFHSATEQEDLLSDIQQAIRLQQQQQQQLQLQLQQQQQQQQLQGAEEPAAPPTAVSPQPGAFLGLDRFLLGPSGGAGEAPGSPDTEQALSALSDLPESLAAEPREPQQPPSPGGLPVSEAPSLPAAQPAAKDSPSSTAFPFPEAGPGEEAAGAPVRGAGDTDEEGEEDAFEDAPRGSPGEEWAPEVGEDAPQRLGEEPEEEAQGPDAPAAASLPGSPAPSQRCFKPYPLITPCYIKTTTRQLSSPNHSPSQSPNQSPRIKRRPEPSLSRGSRTALASVAAPAKKHRADGGLAAGLSRSADWTEELGARTPRVGGSAHLLERGVASDSGGGVSPALAAKASGAPAAADGFQNVFTGRTLLEKLFSQQENGPPEEAEKFCSRIIAMGLLLPFSDCFREPCNQNAQTNAASFDQDQLYTWAAVSQPTHSLDYSEGQFPRRVPSMGPPSKPPDEEHRLEDAETESQSAVSETPQKRSDAVQKEVVDMKSEGQATVIQQLEQTIEDLRTKIAELERQYPALDTEVASGHQGLENGVTASGDVCLEALRLEEKEVRHHRILEAKSIQTSPTEEGGVLTLPPVDGLPGRPPCPPGAESGPQTKFCSEISLIVSPRRISVQLDSHQPTQSISQPPPPPSLLWSAGQGQPGSQPPHSISTEFQTSHEHSVSSAFKNSCNIPSPPPLPCTESSSSMPGLGMVPPPPPPLPGMTVPTLPSTAIPQPPPLQGTEMLPPPPPPLPGAGIPPPPPLPGAGILPLPPLPGAGIPPPPPLPGAAIPPPPPLPGAGIPLPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGAGIPPPPPLPGVGIPPPPPLPGAGIPPPPPLPGAGIPPPPPLPGAGIPPPPPLPRVGIPPPPPLPGAGIPPPPPLPGAGIPPPPPLPGVGIPPPPPLPGVGIPPPPPLPGAGIPPPPPLPGMGIPPAPAPPLPPPGTGIPPPPLLPVSGPPLLPQVGSSTLPTPQVCGFLPPPLPSGLFGLGMNQDKGSRKQPIEPCRPMKPLYWTRIQLHSKRDSSTSLIWEKIEEPSIDCHEFEELFSKTAVKERKKPISDTISKTKAKQVVKLLSNKRSQAVGILMSSLHLDMKDIQHAVVNLDNSVVDLETLQALYENRAQSDELEKIEKHGRSSKDKENAKSLDKPEQFLYELSLIPNFSERVFCILFQSTFSESICSIRRKLELLQKLCETLKNGPGVMQVLGLVLAFGNYMNGGNKTRGQADGFGLDILPKLKDVKSSDNSRSLLSYIVSYYLRNFDEDAGKEQCLFPLPEPQDLFQASQMKFEDFQKDLRKLKKDLKACEVEAGKVYQVSSKEHMQPFKENMEQFIIQAKIDQEAEENSLTETHKCFLETTAYFFMKPKLGEKEVSPNAFFSIWHEFSSDFKDFWKKENKLLLQERVKEAEEVCRQKKGKSLYKIKPRHDSGIKAKISMKT

(19) United States
(12) Patent Application Publication
(10) Pub. No.: US 2012/0082684 A1
(43) Pub. Date: Apr. 5, 2012

(54) DIAGNOSIS AND TREATMENT OF CELL PROLIFERATION AND DIFFERENTIATION DISORDERS BASED ON THE FMN2 GENE

(75) Inventors: Sonia Rocha, Dundee (GB); Angus Iain Lamond, Dundee (GB); Motoharu Ono, Dundee (GB); Kayo Yamada, Dundee (GB)

(73) Assignee: UNIVERSITY COURT OF THE UNIVERSITY OF DUNDEE, Dundee (GB)

(21) Appl. No.: 13/256,315

(22) PCT Filed: Mar. 15, 2010
(86) PCT No.: PCT/GB2010/000448

§ 371 Date: Nov. 8, 2011

(30) Foreign Application Priority Data
Mar. 13, 2009 (GB) 0904382.9

(51) Int. Cl. A61K 39/395; C07H 21/04; A61K 31/7088; C07K 16/18; C12N 15/85; C40B 30/04; C07K 14/47

(52) U.S. Cl. 424/175.1; 506/9; 536/23.5; 530/352; 530/389.8; 530/388.9; 530/391.3; 536/24.33; 536/24.31; 435/320.1; 514/44 R

(57) ABSTRACT

The present invention provides methods for diagnosing cell proliferation and/or differentiation disorders, compounds and methods for treating the same and methods for identifying agents potentially useful in the treatment of cell proliferation and/or differentiation disorders.

### FIELD OF THE INVENTION

The present invention provides methods for diagnosing cell proliferation and/or differentiation disorders, compounds and methods for treating the same and methods for identifying agents potentially useful in the treatment of cell proliferation and/or differentiation disorders.

### BACKGROUND OF THE INVENTION

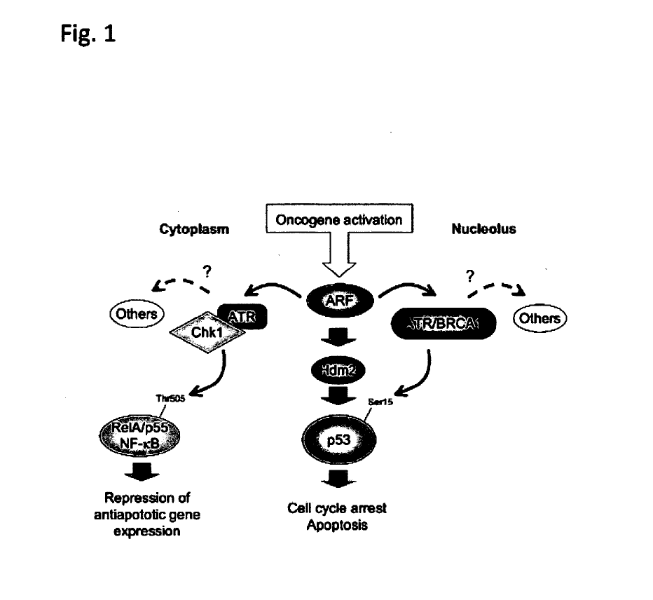

The ARF tumour suppressor is a central component of the cellular defence against oncogene activation in mammals. A high percentage of human leukaemia and melanoma patients have ARF mutations. ARF knock out mice develop tumours with high frequency. ARF activates p53 by stabilizing the protein through inhibition of HDM2, which is an E3 ubiquitin ligase of p53. However, studies on p53 and ARF knock out mice showed there is also an ARF tumour suppressor pathway that is p53 independent (FIG. 1). The ARF protein is concentrated in the nucleus where it is localized to both nucleoli and the nucleoplasm.

### SUMMARY OF THE INVENTION

The present invention is based on the finding that expression of the human Formin-2 (FMN2) gene is dramatically upregulated upon induction of the ARF tumour suppressor and is capable of modulating expression of the cell cycle regulatory protein, p21—an interaction which results in cell cycle arrest.

The present inventors have analysed the effect of ARF on nucleolar protein dynamics using mass-spectrometry-based organellar proteomics and stable isotope labelling, i.e., SILAC, Ong, S. E. et al., Stable isotope labelling by amino acids in cell cultures, silac, as a simple and accurate approach to expression proteomics. Mol. Cell. Proteomics 1, 376-386 (2002). The results show that FMN2 gene expression level is increased by ARF induction, independently of p53. As such, FMN2 genes and/or proteins are considered to serve as useful markers and medicaments for the treatment of a range of cell proliferation and/or differentiation disorders. In addition, FMN2 suppression was observed to have a cytotoxic effect and thus FMN2 may serve as a useful drug target.

The FMN2 gene was first identified by a genome-wide homology search. Thus by searching an EST database for homologues of FMN, probing a mouse cDNA library, and further searching of the human EST database, Leader and Leder (2000) identified a mouse cDNA and a partial human cDNA encoding FMN2. Sequence analysis predicted that the 1,567-amino acid mouse protein and the partial sequence identified for the homologous human protein share approximately 79% identity at their N termini and 90% identity over their C termini. They also have high homology to the Drosophila ‘cappuccino’ protein. The FH1 domain of FMN2 contains 11 proline repeats. Northern blot analysis revealed expression of a 6- to 7-kb FMN2 transcript in all central nervous system tissues and in fetal brain. Whole mount in situ hybridization analysis detected predominant expression of FMN2 in mouse embryonic spinal cord and brain at days 9.5 to 10.5. There are few publications describing analysis of FMN2, e.g. only 15 manuscripts that refer to FMN2 can be found by NCBI PubMed as of March 2010. Most of these publications report analysis of an association between FMN2 and spindle relocation in mouse meiosis.

There are no publications that report association between cancer/tumour suppressor activity and FMN2 in either human or mouse studies.

Accordingly, a first aspect of this invention provides a method of diagnosing a cell proliferation and/or differentiation disorder, or a susceptibility thereto, said method comprising the steps of detecting modulation of Formin2 (FMN2) gene and/or protein expression, wherein modulation of FMN2 gene/protein expression is indicative of a cell proliferation and/or differentiation disorder.

It should be understood that the term modulation encompasses increases and/or decreases in FMN2 gene and/or protein expression, including changes in activity of FMN2 gene products resulting from changes in isoform ratios or post-transcriptional and/or post-translational modification events affecting FMN2 gene products. An increase in the level of FMN2 gene/protein expression may be associated with induction of the ARF tumour suppressor in response to aberrant oncogene activation. As such, one of skill in this field will appreciate that an increase in the level of FMN2 gene/protein expression may be further associated with, for example, cell proliferation and/or differentiation disorders (such as cancer) resulting from the aberrant activation of one or more oncogenes or lack of an active p53.

The term “cell proliferation and/or differentiation disorder” may be taken to encompass a variety of diseases, conditions and/or syndromes including for example neoplastic conditions such as cancer. In this regard, it should be understood that the methods provided by this invention are not limited to the diagnosis of particular types of cancer and as such all forms of carcinoma, sarcoma or lymphoma may be diagnosed thereby. By way of example, it may be possible to diagnose instances of lung, breast, colo-rectal and skin cancers as well as, for example, cancers such as leukaemia. Autoimmune and/or inflammatory disorders such as psoriasis, allergies and the like may also be encompassed within this definition. In addition disorders of meiosis, for example disorders involving the proliferation of germ cells, which may result in infertility or diseases and/or conditions such as hepatitis, liver cirrhosis, renal failure, acute pneumonia, stomach ulcer, cardiac infarction, muscular dystrophy and cerebral infarction may also be encompassed within the term “cell proliferation and differentiation disorders.

Decreases in FMN2 gene and/or protein expression may indicate the presence of, or a susceptibility to, cell proliferation and/or differentiation disorders characterised by aberrant apoptotic events such as, for example, neurodegenerative diseases and acute cellular degenerative diseases and/or conditions such as ischaemic events (for example, stroke, myocardial infarction and the like).

In other embodiments, the methods described herein may be used either to diagnose, or identify, those subjects who are predisposed or susceptible to suffering from cell proliferation and/or differentiation disorders. In subjects who are predisposed or susceptible to cell proliferation and/or differentiation disorders, the level of FMN2 gene and/or protein expression may be elevated.

In one embodiment, modulation of FMN2 gene and/or protein expression may be evaluated relative to the levels of FMN2 gene/protein expression present in reference or control samples derived from healthy individuals (i.e. those not suffering from cell proliferation and/or differentiation disorders). In this way increases and/or decreases in levels of FMN2 gene and/or protein expression may easily be detected.

The term “sample” should be understood as including samples of bodily fluids such as whole blood, plasma, serum, saliva, sweat and/or semen. In other instances “samples” such as tissue biopsies and/or scrapings may be used. In particular biopsies or scrapings from skin, tumours or lymph nodes may be used. In addition, a sample may comprise a tissue or gland secretion and washing protocols may be used to obtain samples of fluid secreted into, for example, the lung. Suitable washing protocols may include broncho-pulmonary lavage procedures. One of skill in this field will apppreciate that each of the samples described herein may yield quantities of FMN2 nucleic acid (i.e. DNA or RNA) and/or FMN2 protein, peptides (or fragments thereof). Furthermore, the methods described herein may comprise the first step of providing a sample from a subject suspected of suffering from a cell proliferation and/or differentiation disorder or who may be at risk of developing a cell proliferation and/or differentiation disorder. As stated, a “reference sample” may be derived from a subject not suffering from a cell proliferation and/or differentiation disorder, exhibiting a “normal” level of FMN2 gene and/or protein expression. The subject from which the sample may be taken, or who may be treated, as described herein, may be a human or animal subject.

One of skill in the art will be familiar with the techniques, which may be used to identify modulated FMN2 gene and/or protein expression, in samples such as those listed above. Such techniques may include, for example, polymerase chain reaction (PCR) based techniques such as real-time PCR (otherwise known as quantitative PCR). In the present case, real time-PCR may used to determine the level of expression of the genes encoding the FMN2 protein. Typically, and in order to quantify the level of expression of a particular nucleic acid sequence, reverse transcriptase PCR may be used to reverse transcribe the relevant mRNA to complementary DNA (cDNA). Preferably, the reverse transcriptase protocol may use primers designed to specifically amplify an mRNA sequence of interest. Thereafter, PCR may be used to amplify the cDNA generated by reverse transcription. Typically, the cDNA is amplified using primers designed to specifically hybridise with a certain sequence and the nucleotides used for PCR may be labelled with fluorescent or radiolabelled compounds.

One of skill in the art will be familiar with the technique of using labelled nucleotides to allow quantification of the amount of DNA produced during a PCR. Briefly, and by way of example, the amount of labelled amplified nucleic acid may be determined by monitoring the amount of incorporated labelled nucleotide during the cycling of the PCR.

Further information regarding the PCR based techniques described herein may be found in, for example, PCR Primer: A Laboratory Manual, Second Edition Edited by Carl W. Dieffenbach & Gabriela S. Dveksler: Cold Spring Harbour Laboratory Press and Molecular Cloning: A Laboratory Manual by Joseph Sambrook & David Russell: Cold Spring Harbour Laboratory Press.

Other techniques, which may be used to determine the level of FMN2 gene expression in a sample, include, for example, Northern and/or Southern Blot techniques. A Northern blot may be used to determine the amount of a particular mRNA present in a sample and as such, could be used to determine the amount of FMN2 gene expression. Briefly, mRNA may be extracted from, a cell using techniques known to the skilled artisan, and subjected to electrophoresis. A nucleic acid probe, designed to hybridise (i.e. complementary to or substantially complementary to) an mRNA sequence of interest—in this case the mRNA encoding the FMN2 protein, may then be used to detect and quantify the amount of a particular mRNA present in a sample.

Additionally, or alternatively, a level of FMN2 gene expression may be identified by way of microarray analysis. Such a method would involve the use of a DNA micro-array, which comprises nucleic acid derived from the FMN2 gene. To identify the level of FMN2 gene expression, one of skill in the art may extract the nucleic acid, preferably the mRNA, from a sample and subject it to an amplification protocol such as, reverse transcriptase PCR to generate cDNA. Preferably, primers specific for a certain mRNA sequence—in this case the sequences encoding the FMN2 gene may be used. The amplified FMN2 cDNA may be subjected to a further amplification step, optionally in the presence of labelled nucleotides (as described above). Thereafter, the optionally labelled amplified cDNA may be contacted with the microarray under conditions, which permit binding with the DNA of the microarray. In this way, it may be possible to identify a level of FMN2 gene expression.

In addition, other techniques such as deep sequencing and/or pyrosequencing may be used to detect FMN2 sequences in any of the samples described above, particularly cell extracts. Further information on these techniques may be found in “Applications of next-generation sequencing technologies in functional genomics”, Olena Morozovaa and Marco A. Marra, Genomics Volume 92, Issue 5, November 2008, Pages 255-264 and “Pyrosequencing sheds light on DNA sequencing”, Ronaghi, Genome Research, Vol. 11, 2001, pages 3-11.

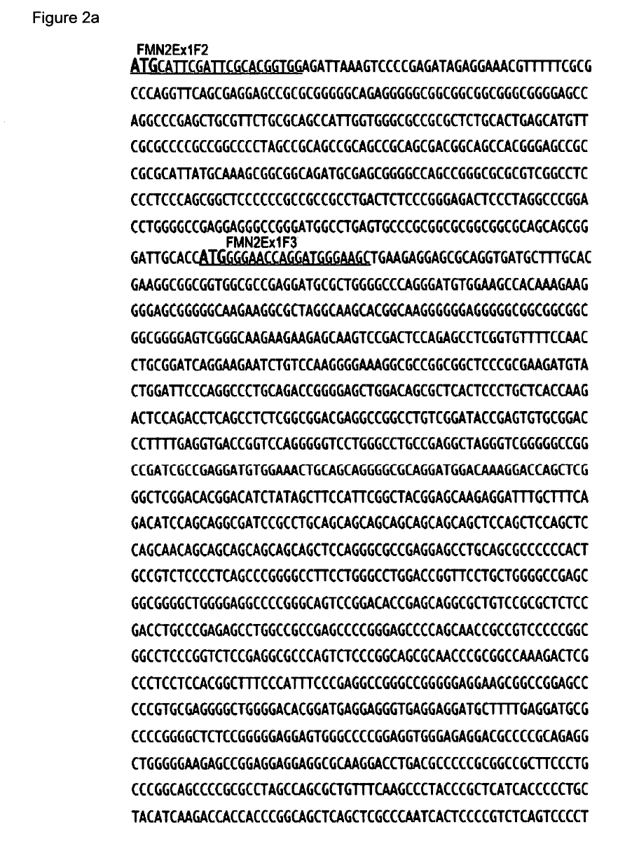

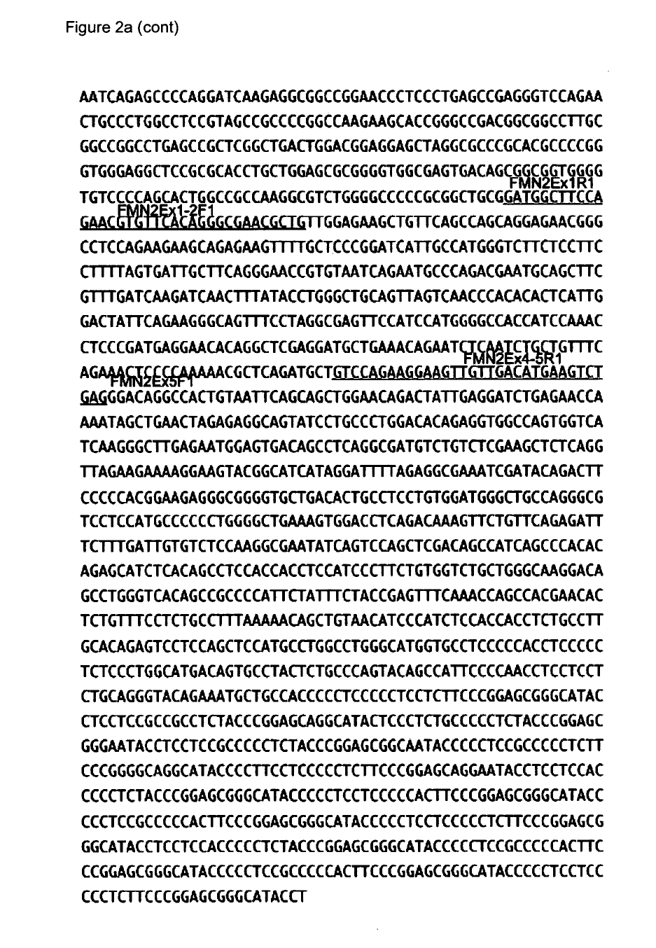

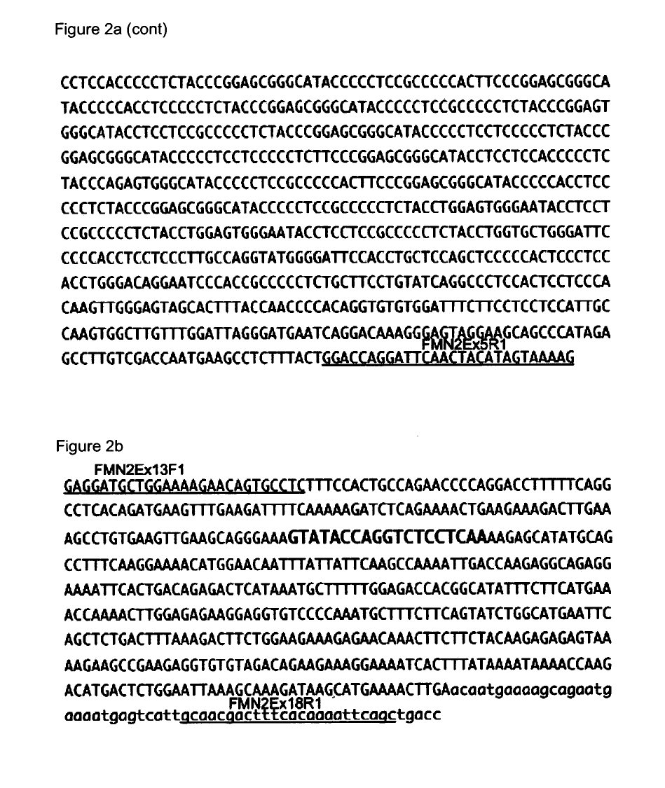

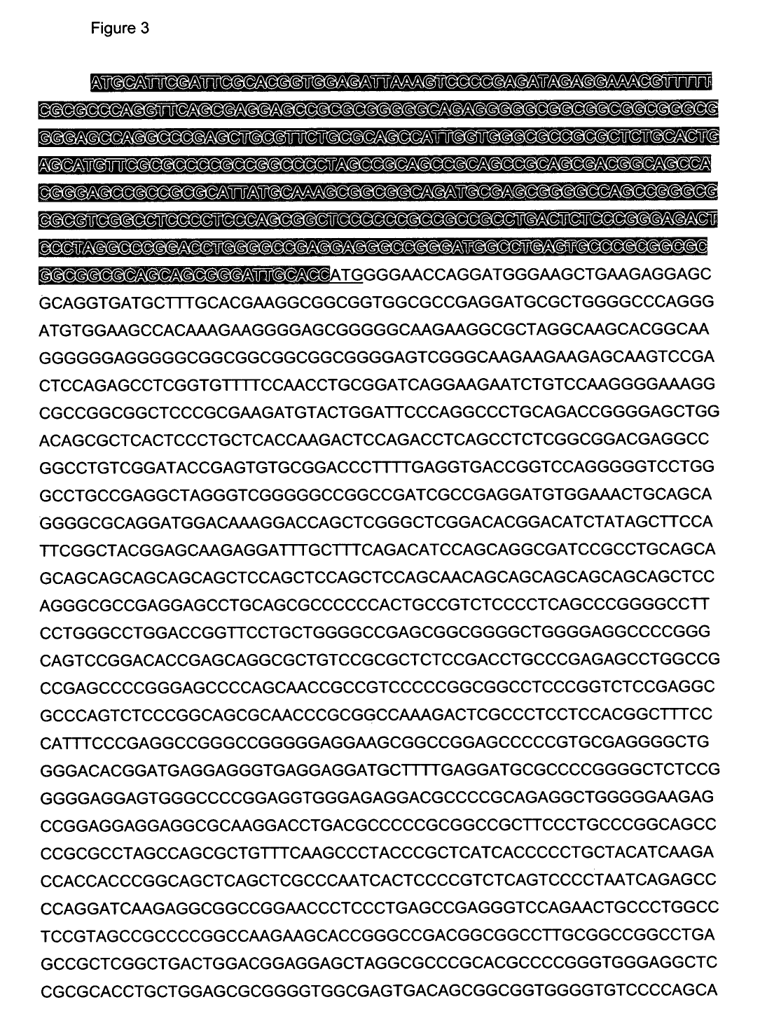

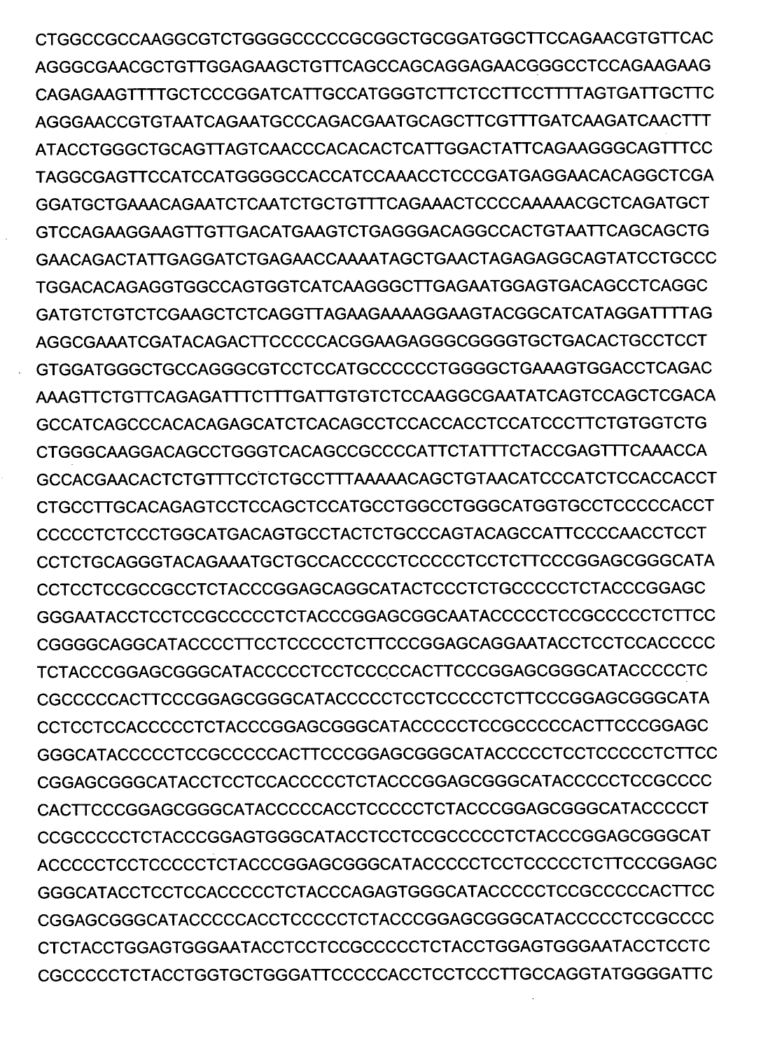

In view of the above, a second aspect of this invention provides the complementary DNA (cDNA) encoding the mature FMN2 protein and a method of obtaining the same. In one embodiment, the cDNA provided by this invention comprises one or more of the sequences shown in FIGS. 2 and 3. In a further embodiment, the FMN2 cDNA sequences are created through transcription and post-transcription modification of the sequence of genomic DNA located on chromosome 1, 1q43 (region: 238321604-238705112). 5′-

The present invention also encompasses cDNA sequences, which are similar to the above sequence and species specific homologues thereof. Thus, the present invention, encompasses sequences which are at level 85%, 90%, 95%, 89% or 99% identical to the identified cDNA sequence.

These homologous sequences may thus correspond to variations linked to mutations within the same species or between species and may correspond in particular to truncations, substitutions, deletions and/or additions of at least one nucleotide. The said homologous sequences may also correspond to variations linked to the degeneracy of the genetic code or to a bias in the genetic code which is specific to the family, to the species or to the variant.

Protein and/or nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J. Mol. Biol. 215(3):403-410; Altschul et al., 1993, Nature Genetics 3:266-272).

In a particularly preferred embodiment, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402).

The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990, Proc. Natl. Acad. Sci. USA 87:2267-2268).

Nucleotide sequence complementary to a sequence of the invention is understood to mean any DNA whose nucleotides are complementary to those of the sequence of the invention, and whose orientation is reversed (antiparallel sequence).

Among representative fragments, those capable of hybridizing under stringent conditions with a nucleotide sequence according to the invention are preferred. Hybridization under stringent conditions means that the temperature and ionic strength conditions are chosen such that they allow hybridization to be maintained between two complementary DNA fragments.

By way of illustration, high stringency conditions for the hybridization step for the purposes of defining the nucleotide fragments described above, are advantageously the following.

The hybridization is carried out at a preferred temperature of 65° C. in the presence of SSC buffer, 1×SSC corresponding to 0.15 M NaCl and 0.05 M Na citrate. The washing steps may be, for example, the following:

2.SSC, 0.1% SDS at room temperature followed by three washes with 1×SSC, 0.1% SDS; 0.5×SSC, 0.1% SDS; 0.1×SSC, 0.1% SDS at 68° C. for 15 minutes.

Intermediate stringency conditions, using, for example, a temperature of 60° C. in the presence of a 5×SSC buffer, or of low stringency, for example a temperature of 50° C. in the presence of a 5×SSC buffer, respectively require a lower overall complementarity for the hybridization between the two sequences.

The stringent hybridization conditions described above for a polynucleotide of about 300 bases in size will be adapted by persons skilled in the art for larger- or smaller-sized oligonucleotides, according to the teaching of Sambrook et al., 1989.

A homologous polypeptide will be understood to designate the polypeptides exhibiting, in relation to the natural polypeptide, certain modifications such as in particular a deletion, addition or substitution of at least one amino acid, a truncation, an extension, a chimeric fusion, and/or a mutation, or polypeptides exhibiting post-translational modifications. Among the homologous polypeptides, those whose amino acid sequence exhibits at least 80%, preferably 90%, homology or identity with the amino acid sequences of the polypeptides according to the invention are preferred. In the case of a substitution, one or more consecutive or nonconsecutive amino acids are replaced by “equivalent” amino acids. The expression “equivalent” amino acid is intended here to designate any amino acid capable of being substituted for one of the amino acids in the basic structure without, however, essentially modifying the biological activities of the corresponding peptides and as will be defined later.

Also encompassed by this invention are splice variants of the primary gene transcripts and the translated FMN2 splice variant proteins, which are encoded thereby. Furthermore, one of skill in this field will readily appreciate that polyadenylation variants and start codon variants, including cDNA sequences encoding the same, may also be included within the scope of this invention.

The cDNA provided by this invention may be used to generate recombinant FMN2 proteins and one of skill will be familiar with the techniques and protocols used to generate recombinant proteins (see for example Gerd Gellissen et al., 2005). In addition, the cDNA may be exploited in assays or microarrays as a means of detecting the expression of the FMN2 gene and/or protein. By way of example, cDNA obtained from samples such as those detailed herein, may be contacted with nucleic acid (i.e. DNA) probes—such as those generated from fragments of genomic DNA, under highly stringent conditions so as to permit binding between DNA probes comprising sequences complementary to those present in the cDNA and the cDNA. When generating cDNA, the nucleotides used for the PCR procedures may be labelled with fluorescent or radiolabelled compounds. In this way cDNA bound to microarrays or the like can easily be detected.

In order to determine the level of FMN2 protein in a sample, immunological techniques exploiting agents capable of binding the FMN2 protein, may be used. The term protein is understood to relate to the whole FMN2 protein, as well as splice variant, isoforms functional fragments, thereof, mutant forms and species specific homologues

In one embodiment, the methods described herein may comprise the step of contacting a substrate (or portion thereof) with a sample to be tested, under conditions which permit the association, interaction, binding and/or immobilisation of any FMN2 protein present in the sample, to said substrate.

Suitable substrates may include, for example, glass, nitrocellulose, paper, agarose and/or plastics. A substrate such as, for example, a plastic material, may take the form of a microtitre plate.

Alternatively, the substrate to be contacted with the sample to be tested may comprise an agent capable of binding the FMN2 protein. Preferably, the agent capable of binding the FMN2 protein is bound to the substrate (or at least a portion thereof). Suitable binding agents may include, for example, antibodies such as monoclonal or polyclonal antibodies and/or other types of peptide or small molecule capable of binding to the FMN2 protein. It is to be understood that this definition applies to all types of binding agent mentioned herein. As such, the substrate (or a portion thereof) may be contacted with the sample to be tested under conditions which permit binding or interaction between the agents capable of binding the FMN2 protein and any FMN2 protein present in the sample.

Any FMN2 protein bound to the substrate or agents capable of binding the FMN2 protein may be detected with the use of a further agent capable of binding the FMN2 protein (referred to hereinafter as the “primary binding agent”). Additionally, or alternatively, the primary binding agents may have affinity for, or bind to FMN2 protein substrate or complexes comprising the FMN2 protein and the above mentioned agents capable of binding the FMN2 protein.

The primary binding agents may be conjugated to moieties, which permit them to be detected (referred to hereinafter as “detectable moieties”). For example, the primary agents may be conjugated to an enzyme capable of reporting a level via a colourimetric chemiluminescent reaction. Such conjugated enzymes may include but are not limited to Horse Radish Peroxidase (HRP) and Alkaline Phosphatase (AlkP). Additionally, or alternatively, the primary binding agents may be conjugated to a fluorescent molecule such as, for example a fluorophore, such as FITC, rhodamine or Texas Red. Other types of molecule, which may be conjugated to binding agents include radiolabelled moieties.

Alternatively, any FMN2 protein bound to the substrate or agents capable of binding the FMN2 protein, may be detected by means of a yet further binding agent (referred to hereinafter as “secondary binding agents”) having affinity for the primary binding agents. Preferably, the secondary binding agents are conjugated to detectable moieties.

The amount of primary binding agent (or secondary binding agent bound thereto) bound to the FMN2 protein, may represent the level of the FMN2 protein present in the sample tested.

In one embodiment, the methods for identifying a level of the FMN2 protein, may take the form of “dip-stick” test, wherein a substrate (or portion thereof) is contacted with a sample to be tested under conditions which permit the binding of any FMN2 protein present in the sample to the substrate or a binding agent bound or immobilised thereto.

In a further embodiment, the methods may take the form of an immunological assay such as, for example, an enzyme-linked immunosorbent assay (ELISA). An ELISA may take the form of a “capture” ELISA wherein, a sample to be tested is contacted with a substrate, and any FMN2 protein present in the sample is “captured” or bound by a binding agent (capable of binding the FMN2 protein) bound or immobilised to the substrate. Alternatively, the sample may be contacted with the substrate under conditions which permit “direct” binding between any FMN2 protein present in the sample and the substrate.

Each of the ELISA methods described above may comprise a “direct” FMN2 protein detection step or an “indirect” identification step. ELISAs involving such steps may be known as “direct” ELISAs or “indirect” ELISAs.

A “direct” ELISA may involve contacting the sample to be tested with a substrate under conditions which permit the binding of any FMN2 protein present in the sample to the substrate and/or a binding agent bound thereto. After an optional blocking step, bound FMN2 protein may be detected by way of an agent capable of binding the FMN2 protein (i.e. a primary binding agent). Preferably, the primary binding agents are conjugated to a detectable moiety.

An “indirect” ELISA may comprise the further step of, after contacting the FMN2 protein with a primary binding agent, using a further binding agent (secondary binding agent) with affinity or specificity for the primary binding agent. Preferably, the secondary binding agent may be conjugated to a detectable moiety.

Other immunological techniques which may be used to identify a level of FMN2 protein in a sample include, for example, immunohistochemistry wherein binding agents, such as antibodies capable of binding the FMN2 protein, are contacted with a sample such as those described above, under conditions which permit binding between any FMN2 protein present in the sample and the FMN2 protein binding agent. Typically, prior to contacting the sample with the binding agent, the sample is treated with, for example a detergent such as Triton X100. Such a technique may be referred to as “direct” immunohistochemical staining.

Alternatively, the sample to be tested may be subjected to an indirect immunohistochemical staining protocol wherein, after the sample has been contacted with a FMN2 protein binding agent, a further binding agent (a secondary binding agent) which is specific for, has affinity for, or is capable of binding the FMN2 protein binding agent, is used to detect FMN2 protein/binding agent complexes.

The skilled man will understand that in both direct and indirect immunohistochemical techniques, the binding agent or secondary binding agent may be conjugated to a detectable moiety. Preferably, the binding agent or secondary binding agent is conjugated to a moiety capable of reporting a level of bound binding agent or secondary binding agent, via a colorimetric chemiluminescent reaction.

The FMN2 protein and isoforms, species specific homologues and fragments can also be detected using functionalised nano-cantilever biosensors and similar devices (Fritz, J. Analyst, 2008, 133, 855-863).

In order to identify the levels of FMN2 protein present in the sample, one may compare the results of an immunohistochemical stain with the results of an immunohistochemical stain conducted on a reference sample. By way of example, a sample which reveals more or less bound FMN2 protein binding agent (or secondary binding agent) than in a reference sample, may have been provided by a subject with, or susceptible to, a cell proliferation and/or differentiation disorder.

Other techniques which exploit the use of agents capable of binding the FMN2 protein include, for example, techniques such as Western blot or dot blot. A Western blot may involve subjecting a sample to electrophoresis so as to separate or resolve the components, for example the proteinaceous components, of the sample. The resolved components may then be transferred to a substrate, such as nitrocellulose. In order to identify any FMN2 protein present in the sample, the substrate may be contacted with a binding agent capable of binding FMN2 protein under conditions which permit binding between any FMN2 protein present in the sample and the agents capable of binding the FMN2 protein.

Advantageously, the agents capable of binding the FMN2 protein may be conjugated to a detectable moiety.

Alternatively, the substrate may be contacted with a further binding agent having affinity for the binding agent(s) capable of binding the FMN2 protein. Advantageously, the further binding agent may be conjugated to a detectable moiety.

In the case of a dot blot, the sample or a portion thereof, may be contacted with a substrate such that any FMN2 protein present in the sample is bound to or immobilised on the substrate. Identification of any bound or immobilised FMN2 protein may be conducted as described above.

In any of the abovementioned techniques, the amount of primary or secondary binding agent detected is representative of, or proportional to, the amount of FMN2 protein present in the sample. Furthermore, the results obtained from any or all of the diagnostic methods described herein may be compared with the results obtained from reference or control samples derived from healthy subjects known not to be suffering from, or susceptible to, a cell proliferation and/or differentiation disorder.

The assays generally described hereinbefore are described in more detail in “The Immunoassay Handbook” 2005 Ed David Wild, Elsevier Ltd, to which the skilled reader is directed.

It will be appreciated that in addition to modulated (i.e. increased or decreased) FMN2 gene and/or protein expression resulting from, for example, the induction of the ARF tumour suppressor, the presence of one or more mutations in the FMN2 gene sequence may also result in aberrant FMN2 gene/protein expression. By way of example, an aberrantly active promoter sequence may cause aberrant expression of the FMN2 gene. Mutations in nucleic acid sequences may take the form of one or more nucleotide additions, deletions, inversions and/or substitutions and upregulated FMN2 expression may result from the presence of one or more of these types of mutation. Accordingly, techniques such as PCR and/or restriction fragment length polymorphism analysis may be used to detect the presence of mutations or particular sequence motifs, known to result in aberrant (i.e. increased or decreased) FMN2 expression.

In one embodiment, a fragment of nucleic acid, which includes a portion of the FMN2 sequence potentially harbouring mutation(s), may be amplified and sequenced, in order to determine whether or not the FMN2 gene comprises a mutation. Alternatively, fragments of this type may be amplified and hybridisation studies carried out using appropriate oligonucleotides and very stringent hybridisation/washing conditions (see for example Sambrook et al, 2001). One of skill in the art will understand that chemically modified oligonucleotides or non-standard oligonucleotides comprising modified nucleobases and/or PNA monomers or those comprising peptide nucleic acid may also be used. In this way, only exactly matching oligonucleotides bind to the amplified fragment in the region or regions comprising the mutation(s).

It may also be appropriate to first amplify a fragment of DNA comprising the sequence which may or may not comprise a mutation(s) and thereafter detecting whether or not the fragment includes the native or mutant sequence by carrying out a further PCR reaction using primers internal to the amplified fragment, in order to detect or otherwise, a mutation(s). Such a technique is commonly known as nested PCR.

Moreover, a mutation may generate a new restriction site or result in the loss of restriction sites present in the wild type or native FMN2 sequence—these changes may easily be detected by RFLP analysis. A fragment which would encompass a mutation which, if present, can first be amplified using appropriate primers and the fragment thereafter subjected to RFLP analysis providing the mutation or native sequence has a restriction site which is not present in the corresponding native or mutant sequence. In accordance with the present invention, the exemplary mutations identified herein result in the generation of new restriction sites which can easily be detected by first amplifying a fragment comprising the mutation and thereafter restricting the fragment obtained using the appropriate restriction enzyme—only a fragment comprising the mutant sequence will be restricted.

The present invention also extends to kits which comprise one or more oligonucleotides/primers for detecting one or more mutations in FMN2 sequences or for more general detection and probing protocols described hereinabove. The kits may also comprise other reagents to facilitate, for example, sequencing, conducting PCR and/or RFLP analysis. Such kits may also comprise instructions for their use to detect one or more mutations in a FMN2 gene and optionally how to interpret whether or not a mutation may lead to development or predisposition to developing any of the aforementioned diseases/conditions. Similarly kits comprising one or more binding agents, such as a FMN2 specific antibody and optionally secondary binding agents, as described herein, may be provided to detect FMN2 protein.

The oligonucleotides/primers of the present invention may also be used in multiplex PCR techniques, known to the skilled addressee, see for example. Kuperstein G, Jack E and Narod S A; Genet Test. 10(1):1-7 (2006), so as to identify mutations in the FMN2 sequence.

Other methods which may be used to detect a level of FMN2 gene and/or protein expression in a sample (including FMN2 splice, start codon and/or polyadenylation variants) include for example, mass spectrometry techniques. By way of example, isolated protein may be cleaved into predictable fragments by either enzymatic digestion, such as with trypsin cleavage, or chemical cleavage, and the mass/charge ratios of the resulting peptides may be detected and/or measured in a mass spectrometer. By comparing the results to those obtained using a reference or control sample (i.e. a sample comprising a normally expressed standard, wild type or non variant FMN2 protein/gene), changes in the levels of FMN2 protein and/or gene expression in different samples can be detected using mass spectrometry. In one embodiment, the reference or control sample may comprise FMN2 genes and/or proteins which are labelled with a chemical tag or through incorporation of a heavy isotope. Further information on these types of protocols may be found in: Aebersold, R. & Mann, M. Mass spectrometry-based proteomics. Nature 422, 198-207 (2003), Ong, S E. et al., Stable isotope labelling by amino acids in cell cultures, silac, as a simple and accurate approach to expression proteomics. Mol. Cell Proteomics 1, 376-386 (2002).

In view of the inventors' finding that inhibition of FMN2 gene and/or protein has a cytotoxic effect (see the detailed description for further discussion and data), a third aspect of this invention provides a compound capable of modulating FMN2 gene and/or protein expression/function, for use in treating cell proliferation and/or differentiation disorders.

A fourth aspect provides the use of compounds capable of modulating FMN2 gene and/or protein expression/function for the manufacture of a medicament for treating cell proliferation and/or differentiation disorders.

A fifth aspect of this invention provides a method of treating a cell proliferation and/or differentiation disorder, said method comprising the steps of administering to a subject in need thereof a compound capable of modulating FMN2 gene and/or protein expression/function.

The term “modulating” should be taken to encompass increases and/or decreases in FMN2 gene/protein expression relative to levels of FMN2 gene and/or protein expression occurring in “normal” or “healthy” systems. One of skill will appreciate that a “normal” or “healthy” system may be a cell derived from, or provided by, a subject not suffering from a cell proliferation and/or differentiation disorder.

Compounds capable of modulating FMN2 gene and/or protein expression may include, for example, DNA or RNA oligonucleotides or chemically modified derivatives of such oligonucleotides and/or antisense oligonucleotides, which are capable of specifically hybridising to FMN2 DNA or RNA. In one embodiment, the oligonucleotides may be RNA molecules known to those skilled in this field as small/short interfering and/or silencing RNA and which will be referred to hereinafter as siRNA. Such siRNA oligonucleotides may take the form of native RNA duplexes or duplexes which have been modified in some way (for example by chemical modification) to be nuclease resistant. Additionally, or alternatively, the siRNA oligonucleotides may take the form of short hairpin RNA (shRNA) expression or plasmid constructs corresponding to, or comprising, the siRNAs described herein (see for example Gregory J. Hannon et al., 2003).

The skilled man will readily understand that antisense oligonucleotides of the type described herein may be used to modulate (for example, inhibit, down-regulate or substantially ablate) the expression of any given gene. Furthermore, the modulation of gene expression or function may also have a similar or “knock-on” effect upon the expression and/or function of any proteins encoded by the modulated gene. Accordingly, the (antisense) oligonucleotides provided by this invention may be designed to modulate the expression and function of the FMN2 gene and/or its protein product.

By analysing native or wild-type FMN2 sequences and with the aid of algorithms such as BIOPREDsi, one of skill in the art could easily determine or computationally predict nucleic acid sequences that have an optimal knockdown effect for these genes (see for example: http://www.biopredsi.org/start.html). Accordingly, the skilled man may generate and test an array or library of different oligonucleotides to determine whether or not they are capable of modulating the expression or function of the FMN2 genes and/or proteins.

In one embodiment the antisense oligonucleotide may be an siRNA molecule directed against the FMN2 mRNA and having the sequence:

5′-GUAUACCAGGUCUCCUCAA-3′

In view of the above, the antisense oligonucleotides and/or siRNA molecules described herein may be used (i) to treat cell proliferation and/or differentiation disorders, (ii) in the manufacture of medicaments for treating the same or (iii) in methods of treating subjects suffering from cell proliferation and/or differentiation disorders.

As an alternative to siRNA control of gene expression, targeted modulation of gene expression may be effected by specific snoRNA constructs which are designed to modulate expression of FMN2. The methods for achieving this are described for example in PCT/GB2008/003211.

In other embodiments, antibodies capable of binding to the FMN2 protein may be useful in the treatment of cell proliferation and/or differentiation disorders. Antibodies which block or neutralise the function of the FMN2 protein or which block interaction between it and other proteins (for example the cell cycle protein, p21) may be particularly useful.

The techniques used to generate polyclonal and/or monoclonal antibodies (mAbs) are well known and described in “The Immunology Handbook” mentioned hereinabove and can easily be exploited to generate antibodies specific for the FMN2 protein or fragments thereof, which competitively compete with any native p21.

Other compounds useful in the treatment of cell proliferation and/or differentiation disorders may include for example, proteins, peptides, amino acids, carbohydrates and other small organic molecules. By way of example, compounds capable of interfering with or preventing interactions between FMN2 and the cell cycle protein, p21, may be particularly useful. Such compounds may take the form of the whole p21 protein or fragments thereof.

Additionally, or alternatively, the compounds capable of modulating FMN2 gene and/or protein expression/function may specifically bind to particular regions of the FMN2 protein identified as being important to FMN2 function and/or interactions between the FMN2 protein and others involved in regulation of the cell cycle (for example p21). In some instances, the compounds capable of modulating FMN2 gene and/or protein expression/function, may indirectly affect the expression and/or function of other genes and/or proteins such as, for example p53 and/or Hdm2. Other compounds may have no affect upon the expression and/or function of other genes and/or proteins.

A sixth aspect of this invention provides a method of identifying or obtaining agents which modulate the expression of the FMN2 gene and/or protein, said method comprising the steps of contacting the FMN2 gene or protein with a test agent and detecting any modulation of FMN2 gene and/or protein expression/function.

One of skill in this field will appreciate that a method such as that described in this sixth aspect of the invention may be conducted in systems such as, for example, cell based or cell free systems, modified to include the FMN2 gene and/or to express the FMN2 protein. By way of example, cells may be transfected with nucleic acid comprising the FMN2 gene. In one embodiment, the nucleic acid may take the form of a vector (for example a plasmid or expression cassette well known in the art).

In one embodiment, the results obtained from the methods described above may be compared to those obtained from a control method in which the FMN2 gene and/or protein have not been contacted with a test agent. In this way, it may be possible to determine whether or not said agent is capable of modulating the expression of the FMN2 gene or protein. Where the level of FMN2 gene or protein expression is less or greater than the level of expression detected in the control method, the test agent may be useful as a modulator of FMN2 gene and/or protein expression. Where the level of expression is the same as that observed in the control methods, the test agent is most likely not capable of modulating the expression of the FMN2 gene or protein.

Suitable test agents may take the form of nucleic acids, for example the antisense oligonucleotides described above, proteins, peptides, amino acids, antibodies (and fragments thereof), carbohydrates and other small organic molecules.

In a seventh aspect, the present invention provides pharmaceutical compositions comprising any of the compounds described above (for example, oligonucleotides, antibodies, small organic compounds, p21 protein or fragments thereof) and/or any of the agents identified by the methods provided by the sixth aspect of this invention and which are capable of modulating the expression or function of the FMN2 genes/proteins, in association with a pharmaceutically acceptable excipient, carrier or diluent. Such compositions may find application in, for example, the treatment of cell proliferation and/or differentiation disorders such as those described above.

Preferably, the pharmaceutical compositions provided by this invention are formulated as sterile pharmaceutical compositions. Suitable excipients, carriers or diluents may include, for example, water, saline, phosphate buffered saline, dextrose, glycerol, ethanol, ion exchangers, alumina, aluminum stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycon, sodium carboxymethylcellulose, polyacrylates, waxes, polyethylene-polypropylene-block polymers, polyethylene glycol and wool fat and the like, or combinations thereof.

Said pharmaceutical formulation may be formulated, for example, in a form suitable for oral, parenteral or topical administration. Pharmaceutical compositions formulated for topical administration may be presented as an ointment, solution or a suspension in an aqueous or non-aqueous liquid, or as an oil-in-water liquid emulsion.

As stated, the present invention is based upon the observation that FMN2 overexpression is upregulated by ARF induction at the mRNA level. As such, the FMN2 gene possesses an ARF responsive promoter which may itself find application in the field of gene therapy. As such, the present invention also extends to methods of treating prophylactically or therapeutically any of the aforementioned diseases/conditions by administering to a patient suffering or predisposed to developing any of said aforementioned diseases a DNA construct comprising an gene sequence or fragment thereof, under control of the ARF responsive FMN2 promoter, which gene sequence or fragment thereof is capable of expressing one or more copies of a protein potentially useful in the treatment of said disease and/or condition, whereby expression of said one or more copies said protein treats or ameliorates said disease(s)/condition(s).

Typically, the protein potentially useful in the treatment of the cell proliferation and/or differentiation disorder will be administered to a subject in the form of a recombinant molecule comprising said gene sequence under appropriate transcriptional/translational controls to allow expression of the potentially useful protein when administered to a subject. It will be appreciated that the gene sequence or fragment may be under control of a suitable promoter, such as a constitutive and/or controllable promoter. Convenient promoters include the native FMN2 promoter—referred to hereinafter as an ARF responsive promoter. The ARF responsive promoter is located within chr1:238,139,341-238,321,875 (182468 bases). The inventors have further characterised the promoter region as described in the examples section. In this regard a region approximately 2 Kb upstream of the FMN2 start codon has been shown to possess the promoter and the ability to be ARF responsive. The protein to be expressed may be a cytotoxic agent, such as cytosine deaminase and herpes simplex virus thymidine kinase etc. (Cestmir Altaner, 2008) which will only be expressed as a result of ARF being present, such as in cancer cells. This may also be used prophylactically to be administered to subjects who may be predisposed to developing the aforementioned diseases/conditions, such that expression occurs only once ARF activations occurs.

The present invention also therefore provides a recombinant molecule comprising a gene sequence encoding a protein or fragment thereof under control of a FMN2 ARF responsive promoter, for use in therapy. The recombinant molecule may be in the form of a plasmid, phagemid or viral vector. Furthermore, recombinantly expressed, or chemically synthesised FMN2 protein, or functionally important fragments thereof, may be produced and used as a treatment or prophylatic measure for cell proliferation and/or differentiation disorders associated with a decrease in FMN2 expression, or when a subject expressed mutant or aberrant forms of FMN2.

Many different viral and non-viral vectors and methods of their delivery, for use in gene therapy, are known, such as adenovirus vectors, adeno-associated virus vectors, retrovirus vectors, lentiviral vectors, herpes virus vectors, liposomes, DNA vaccination and the like.

By way of example, a gene potentially useful in the treatment of a disorder such as, for example, a cell proliferation and/or differentiation disorder, in particular a cell proliferation and/or differentiation disorder involving ARF induction, may be ligated to the FMN2 ARF responsive promoter described herein. The ARF responsive promoter may be ligated to the potentially useful gene with the aid of a vector engineered to comprise the FMN2 ARF responsive promoter sequence. Suitable vectors may include, for example, plasmids or nucleic acid cassettes. The vectors may then be used (perhaps as medicaments) to treat the abovementioned disorders. One of skill will appreciate that any method of treating a cell proliferation and/or differentiation disorder by gene therapy, may comprise the steps of administering a gene/FMN2 ARF responsive promoter complex to a subject in need thereof, such that the gene becomes expressed in the subject under the control of the FMN2 ARF responsive promoter. One of skill will appreciate such that the methods and medicaments described herein are particularly useful where the disorder to be treated is associated with induction of the ARF tumour suppressor. It will also be appreciated that the medicaments and methods described herein may involve administering the nucleic acid constructs directly to the diseased tissue (for example by injection into a tumour) or administration by any of the other routes described above. The ARF responsive FMN2 promoter may comprise the ARF responsive element fused to an alternative promoter, such as the CMV promoter or the like.

### DETAILED DESCRIPTION

The present invention will now be described in detail with reference to the following Figures which show:

FIG. 1. Schematic drawing of ARF tumour suppressor signaling. The ARF tumour suppressor is a central component of the cellular defence against oncogene activation. A high percentage of leukaemia and melanoma patients have ARF mutations. ARF knock out mice develop tumours with high frequency. ARF activates p53 by stabilizing the protein through inhibition of HDM2, which is the E3 ubiquitin ligase of p53. However, studies on p53 and ARF knock out mice showed there is also an ARF tumour suppressor pathway that is p53 independent;

FIG. 2a The sequence encoding N-terminal region of FMN2. There are 2 possible start codons in Exon1 and these are shown as bold;

FIG. 2b The sequence encoding C-terminal region of FMN2. The sequence which we targeted by siRNA is shown as bold;

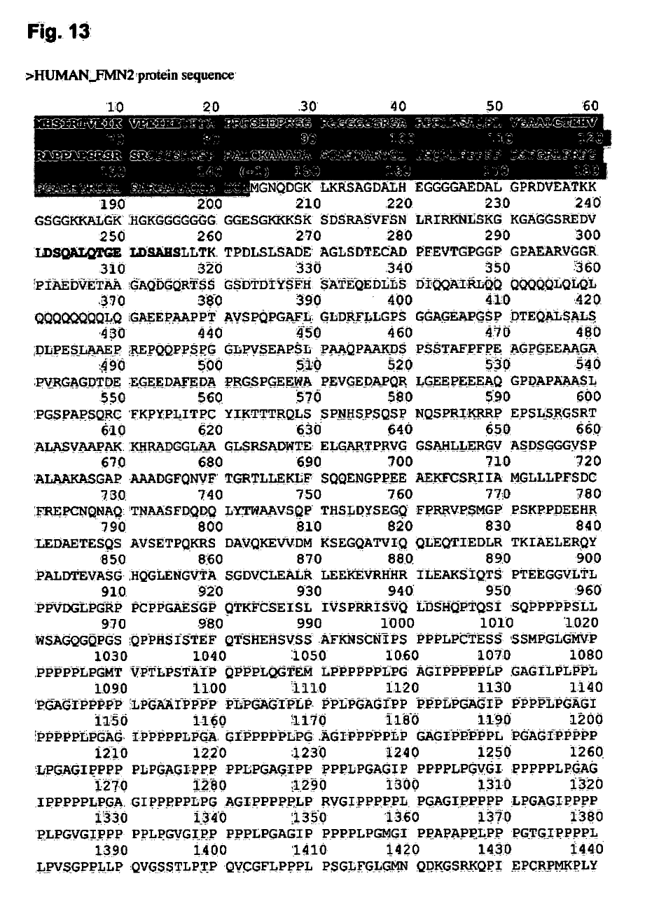

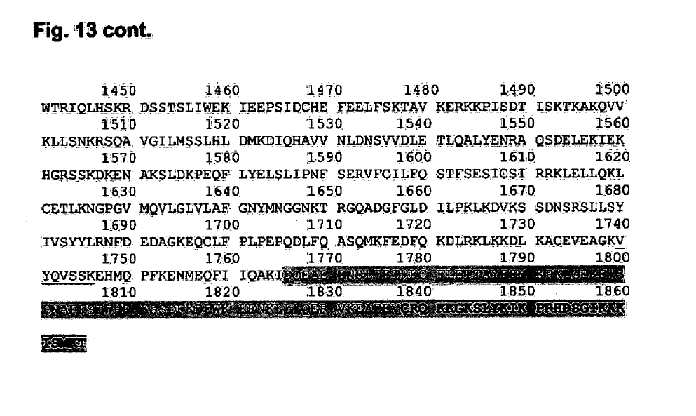

FIG. 3. The complete cDNA sequence of FMN2; Upper case letter shows FMN2 protein coding region (Protein sequence is in FIG. 13). Lower case letter shows 3′UTR from human genome database. Predicted start codon by human genome project is shown underlined. A new start codon and coding sequence that was discovered by this study is shown in grey shading.

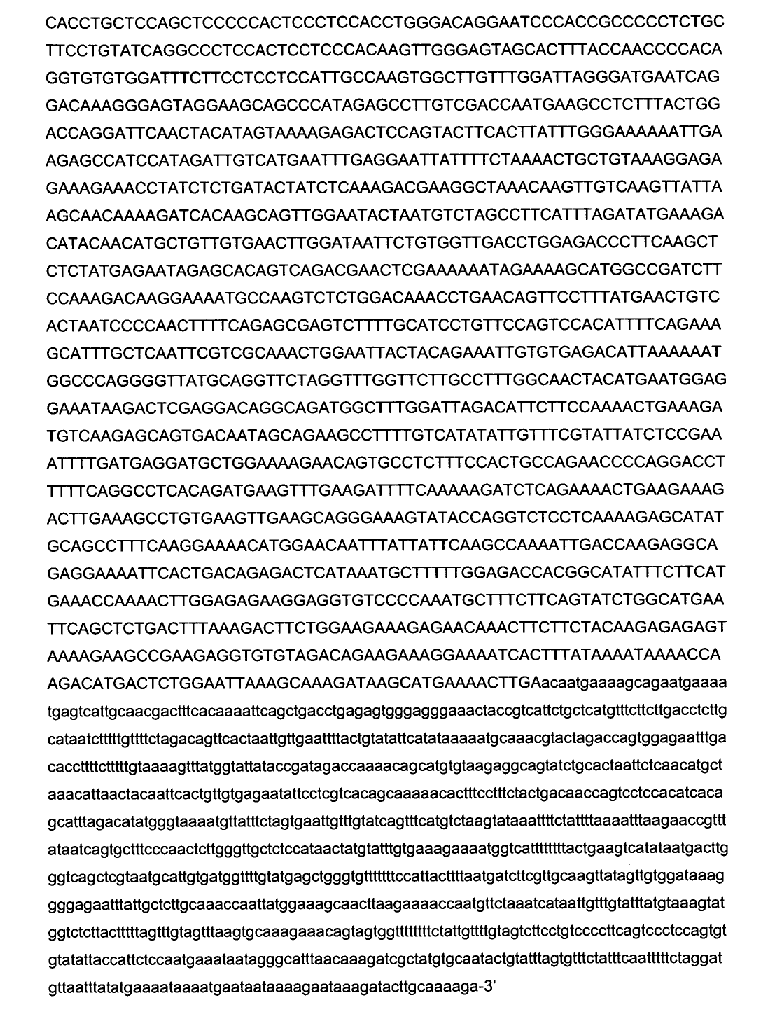

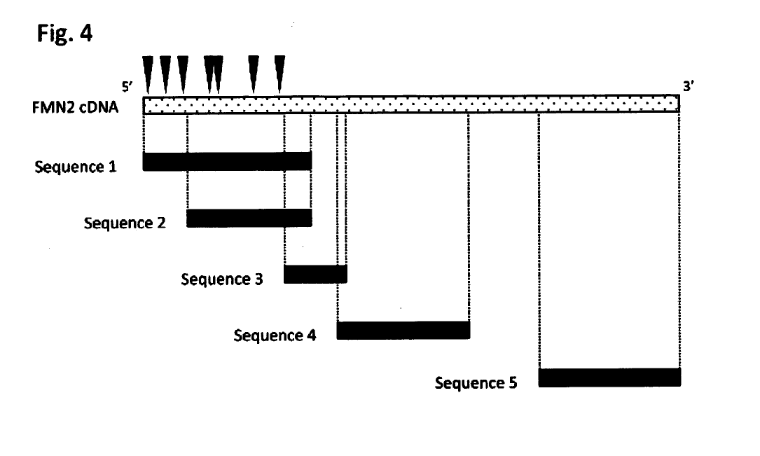

FIG. 4. Schematic drawing of FMN2 sequences. FMN2 gene positions (From 1 stATG-TGA). (ATG) chr1:238,321,604-238,704,077 (TGA). Sequence 1: from 1^{st} ATG of Exon1 to Exon1 and Exon2 junction using primer FMN2Ex1F2 and FMN2Ex1-2R1. Sequence 2: from 2^{nd} ATG of Exon1 to Exon1 and Exon2 junction using primer FMN2Ex1F3 and FMN2Ex1-2R1. Sequence 3: from Exon1 and Exon2 junction to the end of Exon4 using primer FMN2Ex1-2F1 and FMN2Ex4-5R1 (from reported start codon). Sequence 4: Entire Exon5 using primer FMN2Ex5F1 and FMN2Ex5R1 (from reported start codon). Sequence 5: from the beginning of Exon13 to Exon18 including stop codon using primer FMN2Ex13F1 and FMN2Ex18R1 (from reported start codon). Arrowheads indicate positions which we detected by mass spectrometry;

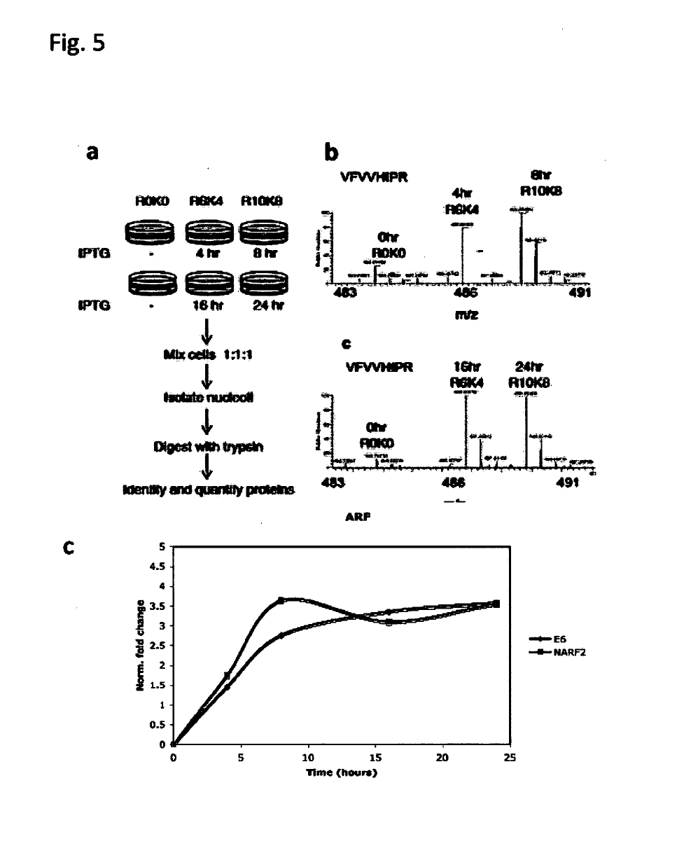

FIG. 5. Determination of nucleolar protein dynamics. a. The proteomes in three cell populations are encoded by incorporation of stable isotope derivatives of arginine (SILAC method). Cells are metabolically labeled with Arg0, Arg6 and Arg10 for at least five cell doubling and are then treated with IPTG for 0, 4, and 8 hours or 0, 16 and 24 hours to induce p14ARF, respectively. Cells are mixed and nucleoli purified and analysed by mass spectrometry. The analysis is repeated three times with a common zero point. b. c. Spectra of peptides of p14ARF, indicating increasing amounts p14ARF recruited to nucleolus. d. Dynamic profile of p14ARF. Y axis is in units of normalized fold change of p14ARF.

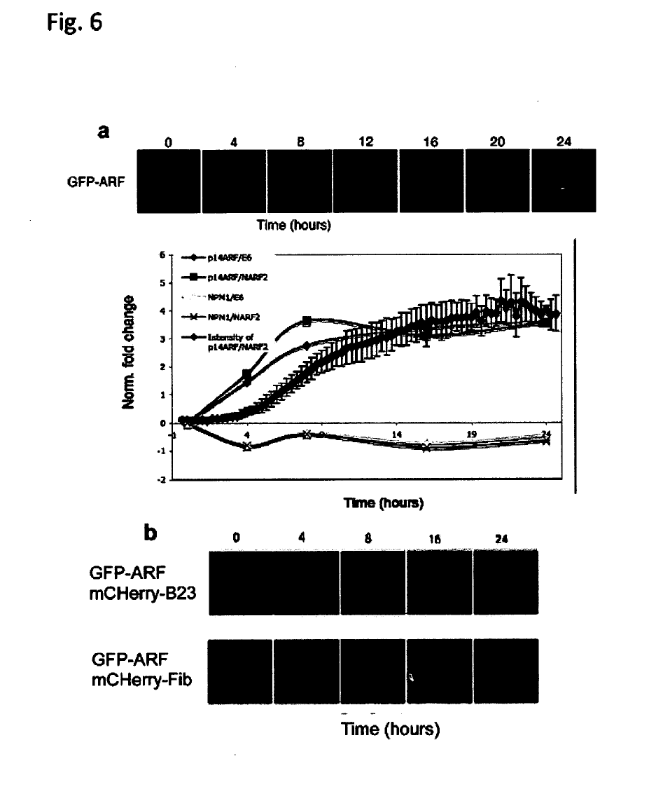

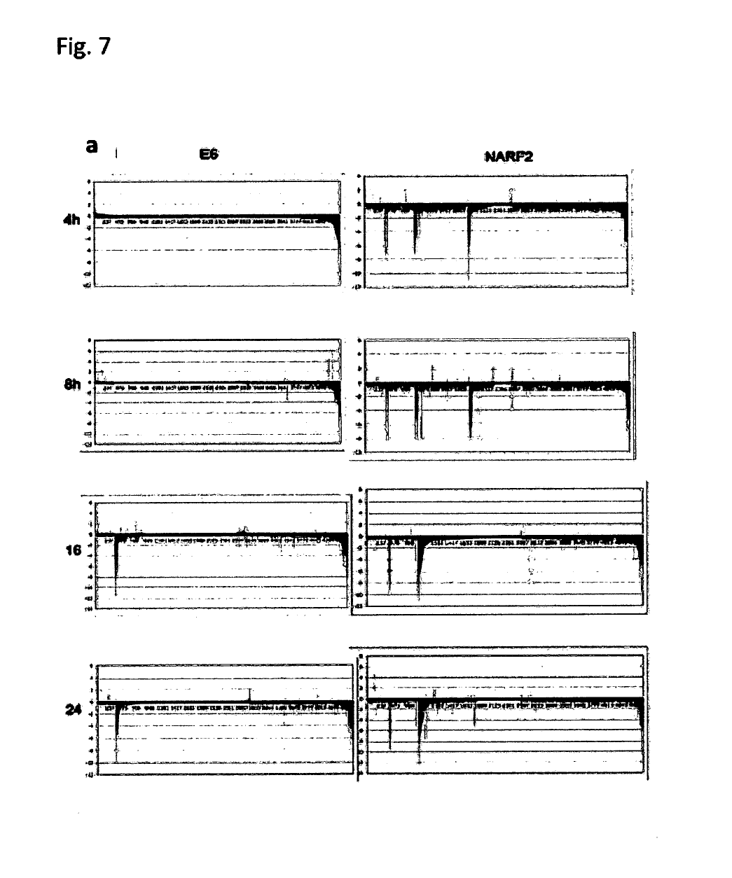

FIG. 6. Comparison of different methods to measure nucleolar protein dynamics. a. Live U2OS cells expressing GFP-ARF during doxycyclin induction (5 μg ml⁻¹) and imaged for 24 hours. The changes in the intranucleolar. GFP fluorescence signals (blue curves) were compared to the changes in levels of the induced p14ARF detected in isolated nucleoli by SILAC. Error bars are s.d. from fluorescence measurement on five individual cells in each case. b. Expression pattern of transiently transfected nucleolar proteins and GFP-ARF in U2OS cells.

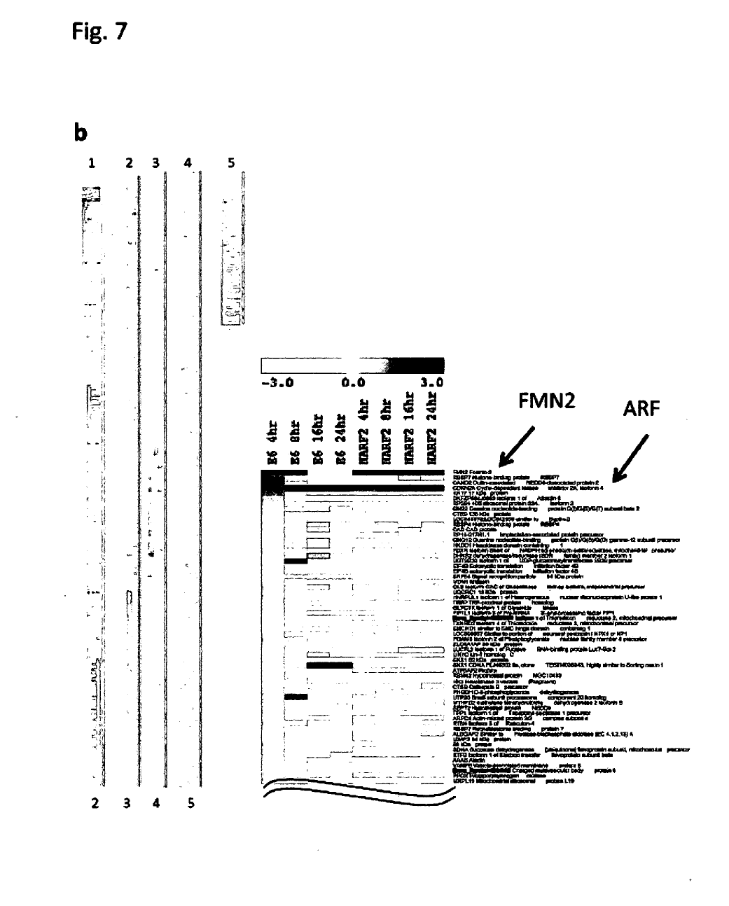

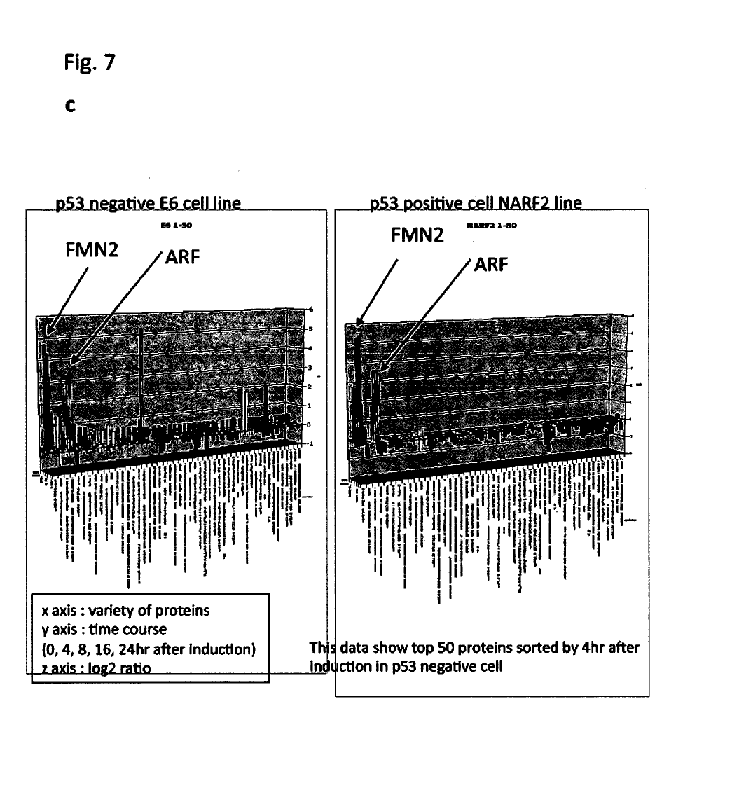

FIG. 7. Dynamic profiles of nucleolar proteins. a. All proteins showing change from first to last time points. b. Hierarchical clustering of 3500 proteins using fold change data. c. The time course change of the top 50 proteins showing the largest percentage changes are shown by time point 4 hr after induction in p53 negative E6 cells.

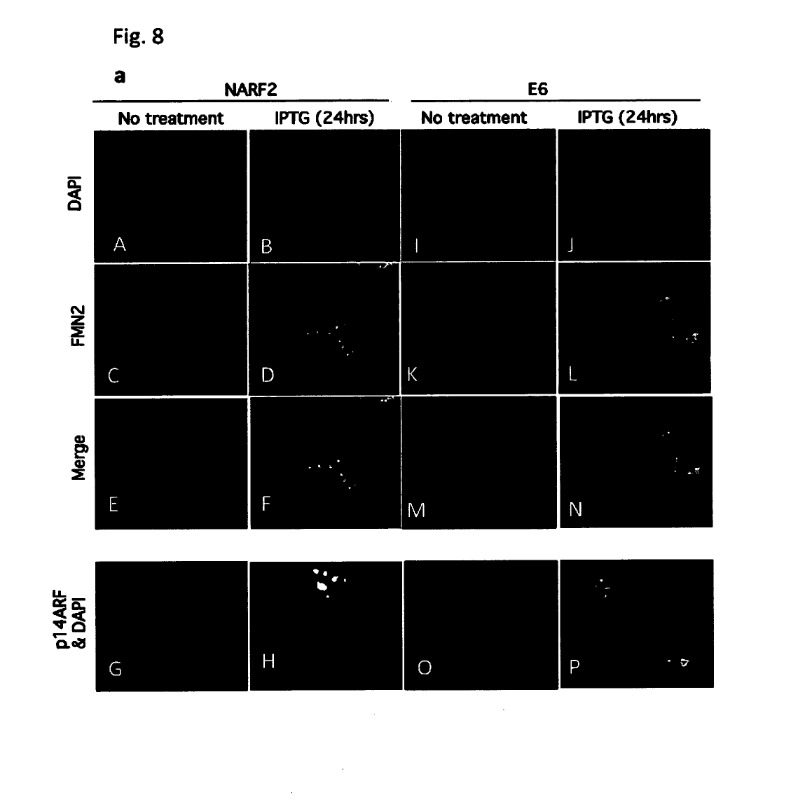

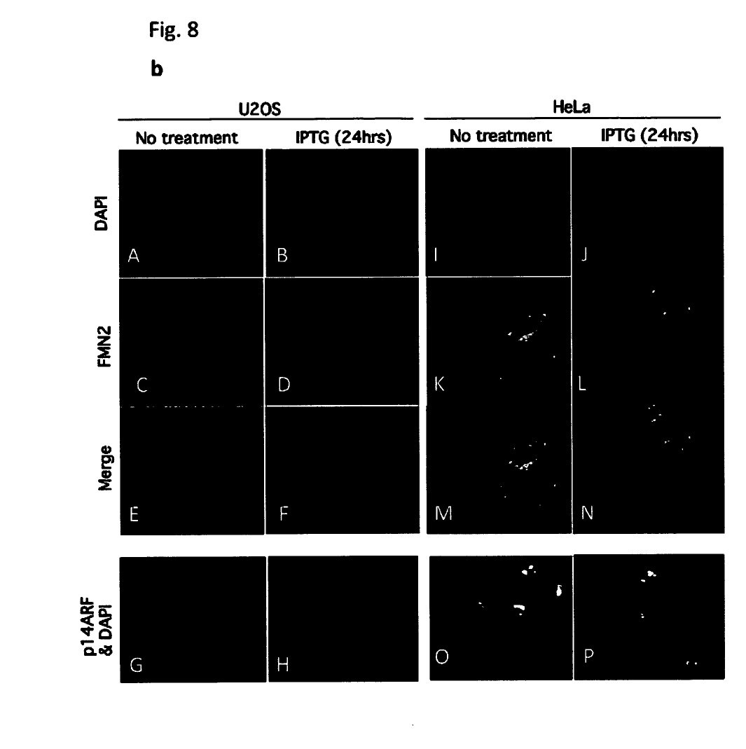

FIG. 8. Expression pattern of the FMN2. NARF2 (A-H), E6 (L-P) cells a. and U2OS (A-H) HeLa (L-P) cells b. were fixed and stained with DAPI for DNA (blue), and anti-FMN2 antibody or anti-p14ARF antibody (green). Cells were incubated for 24 hours with or without IPTG to induce exogeneous p14ARF expression.

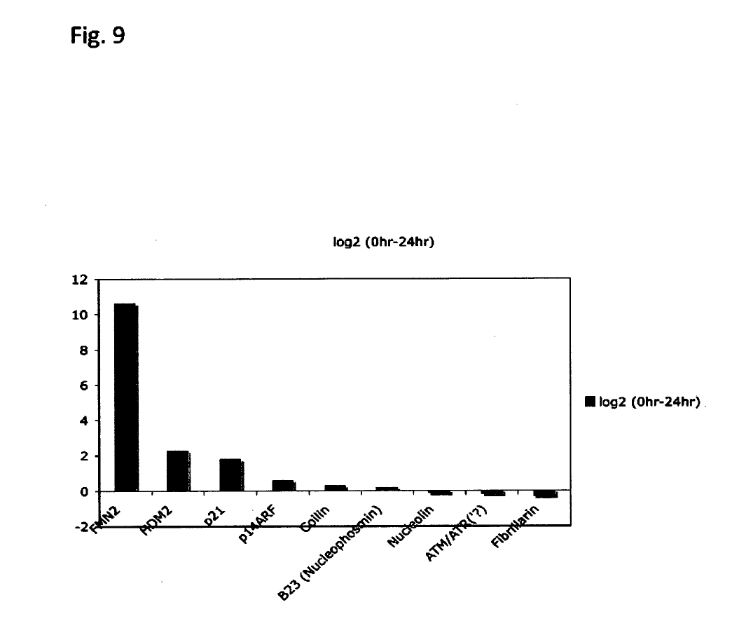

FIG. 9. Microarray analysis during ARF activation. NARF2 cells were incubated for 24 hours with IPTG to induce exogeneous ARF expression. We chose FMN2, HDM2, p21, p14ARF, coilin, B23, Nucleoln, ATM/ATR, and Fibrillarin from our final datasets and showed as a log 2 ratio.

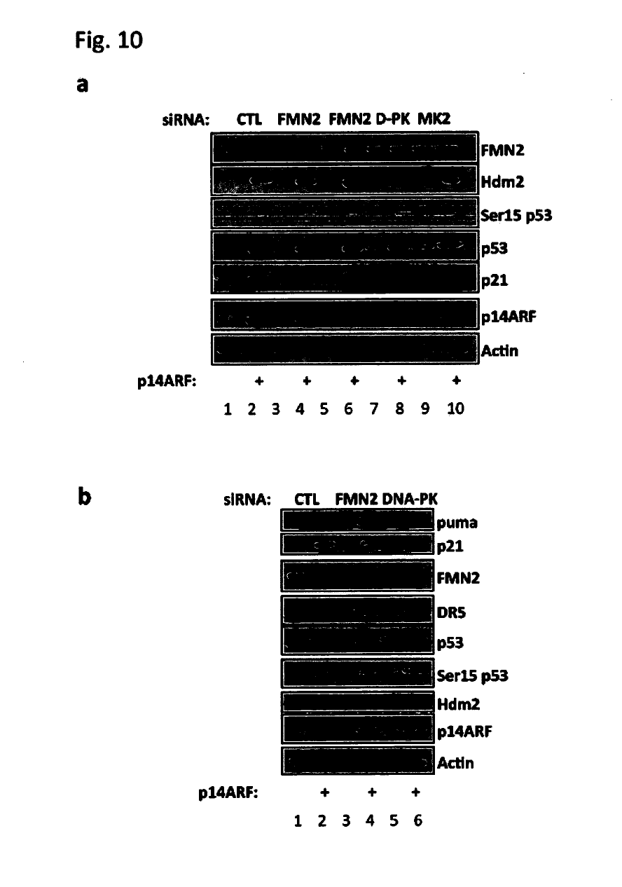

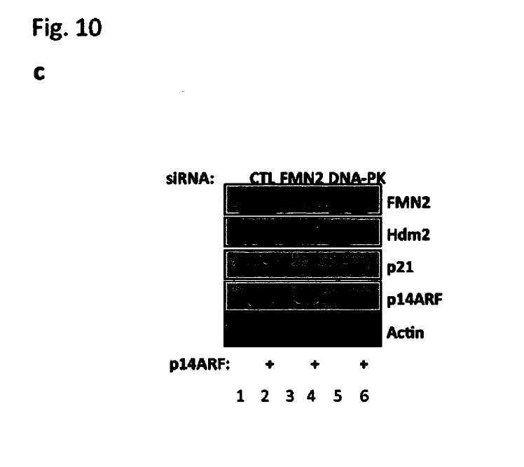

FIG. 10. siRNA approach for FMN2. NARF2 cells were treated with Control, DNA-PK, or FMN2 siRNA and harvested 48 hours with or without 24 hours IPTG induction.

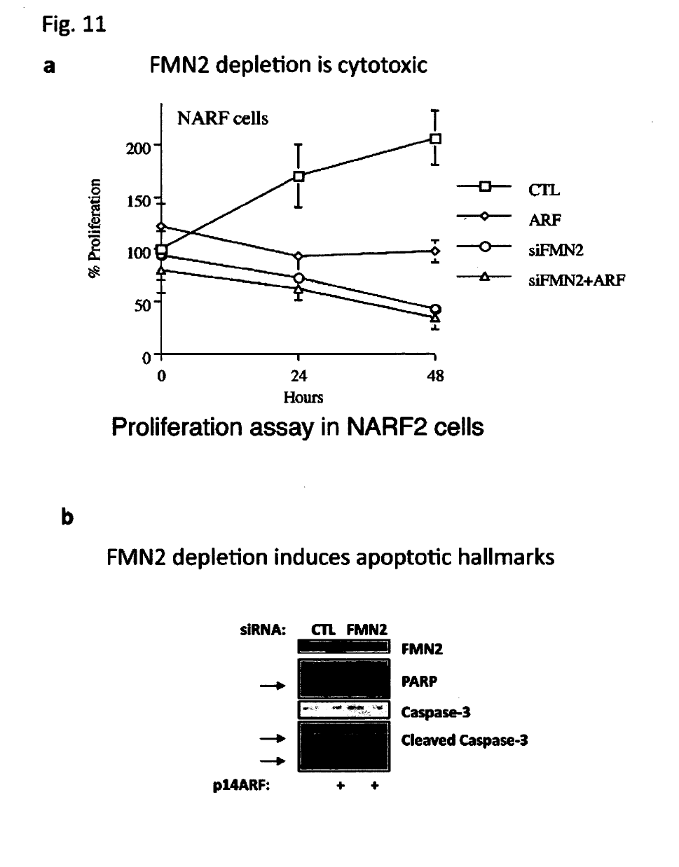

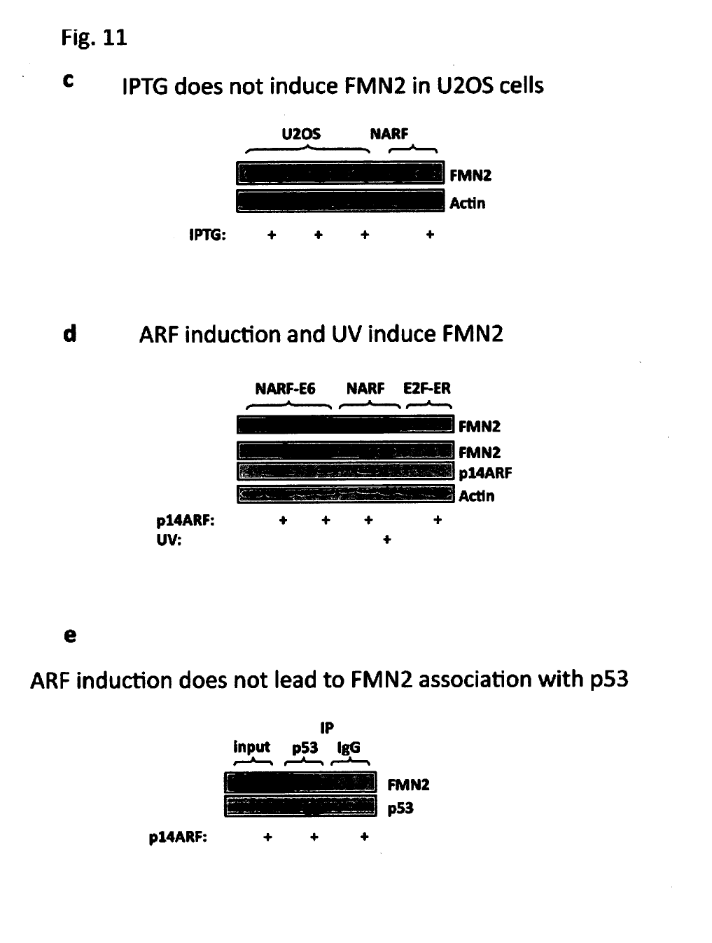

FIG. 11. a-b. effects of FMN2 depletion. c. Effects of IPTG and d. ARF induction and UV on FMN2 induction. e. ARF induction and effect on FMN2 association with p53.

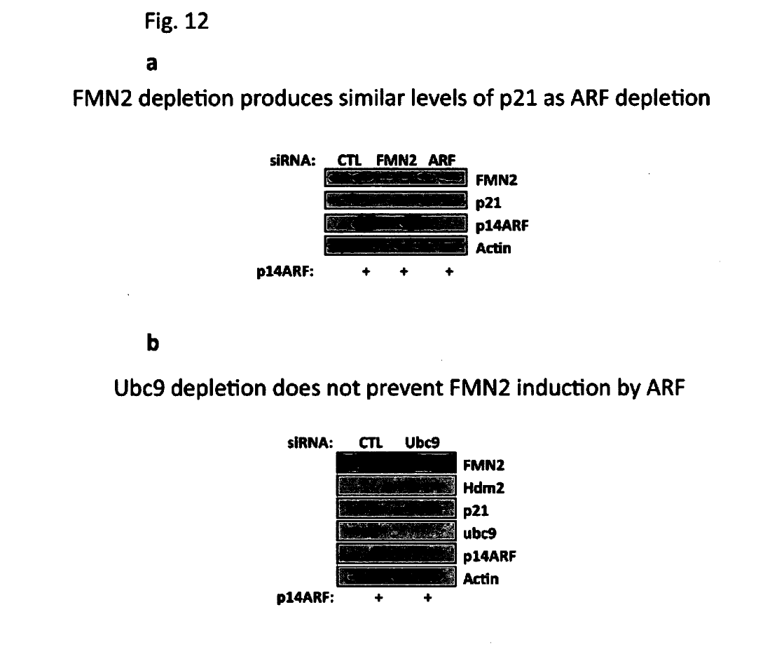

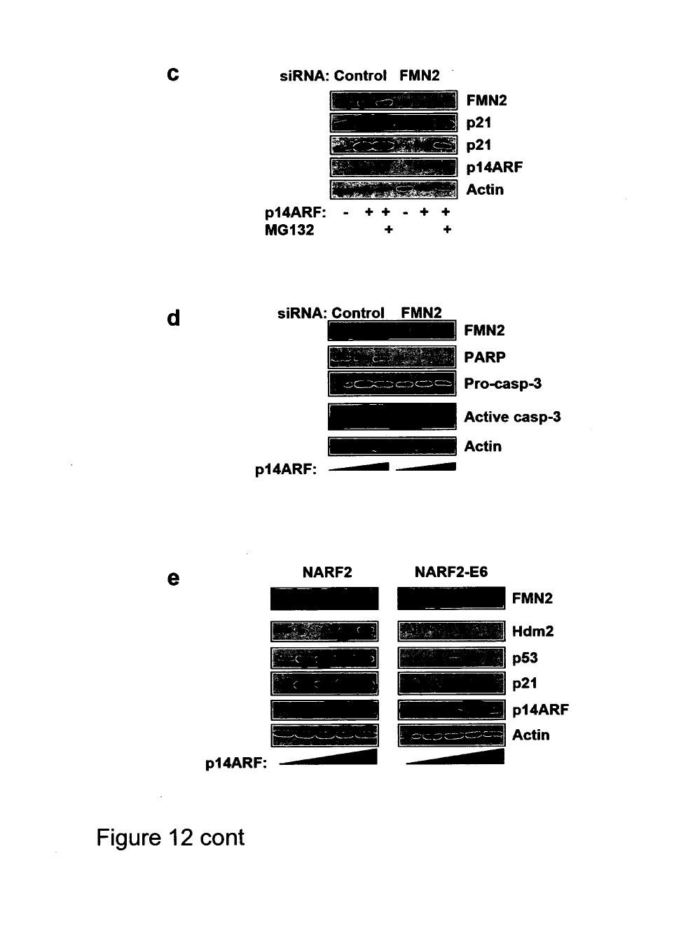

FIG. 12. a. Effects of FMN2 depletion on p21 expression. b. Effect of Ubc9 depletion on FMN2 induction by ARF. c-e. siRNA approach for FMN2. FIG. 12c shows that FMN2 stabilizes p21 by preventing the proteosome pathways.

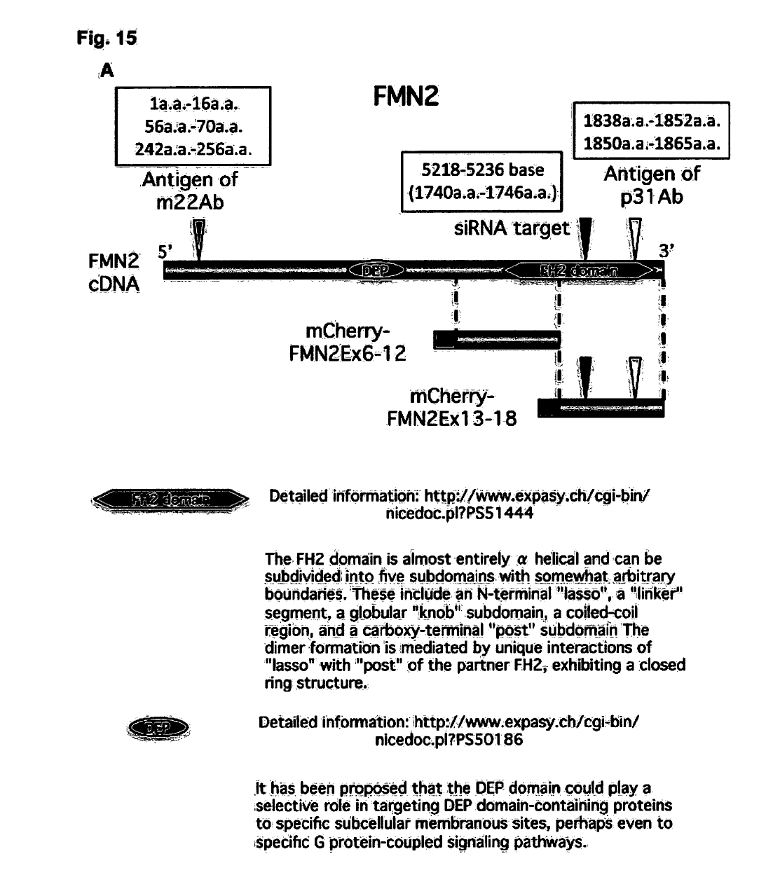

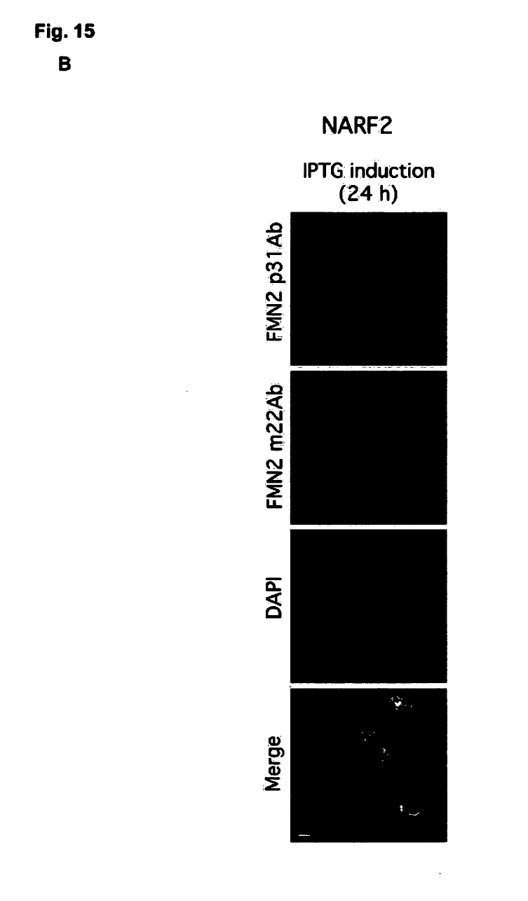

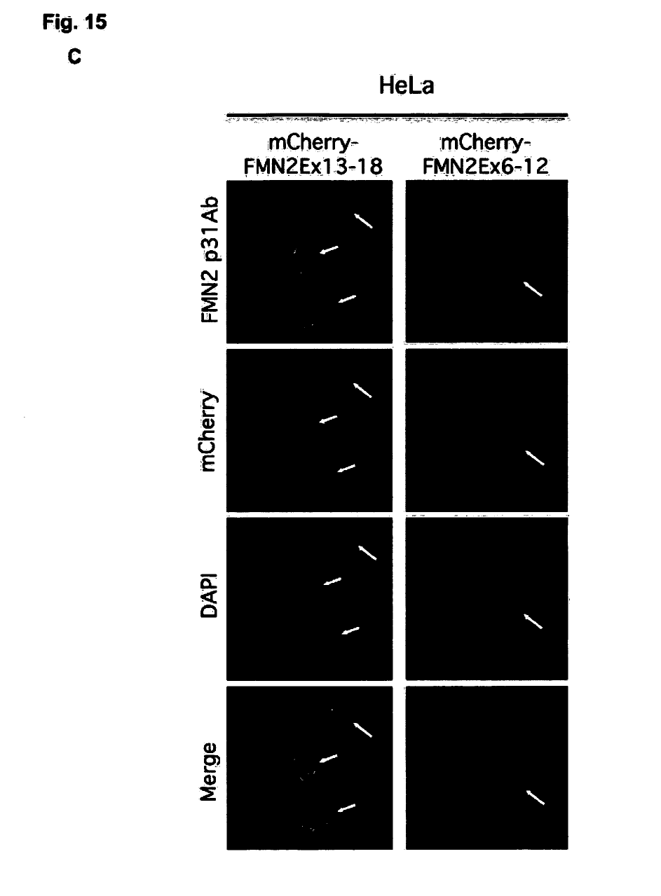

FIG. 13 shows protein sequence of FMN2 and the antigen regions for antibody production. Human FMN2 amino acid sequence is shown. New additional sequence which was identified in this study is shown in grey (1a.a.-143a.a.). Antigen sequences are shown in bold (see also methods and FIG. 15A). The antigen region of commercially available FMN2 anti-body (Abnova) was also shown in grey (see also methods).

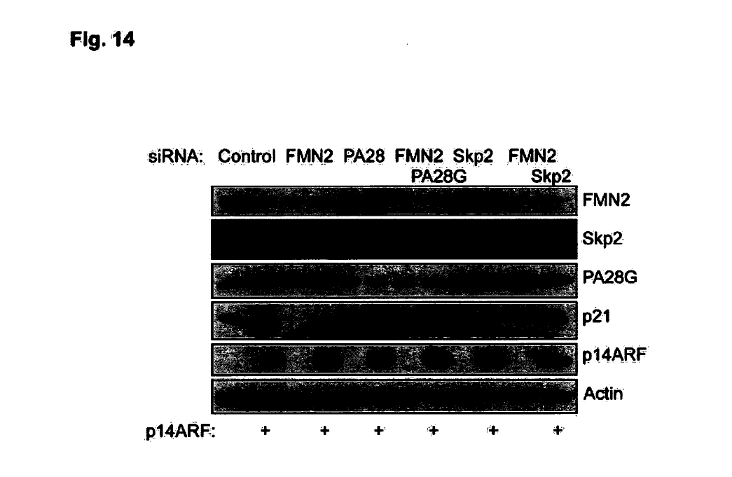

FIG. 14 shows the results of a Western blot showing the rescue of FMN2 depletion with siRNA for PA28γ and Skp2;

FIG. 15A shows a schematic representation of the FMN2 protein and domains. The schematic shows the region of the protein used to generate the monoclonal antibody (m22Ab) and the polyclonal antibody (p31Ab). The schematic also shows the regions of the protein from which the two truncated conjugated polypeptides mCherry-FMN2 Ex6-12 and mCherry FMN2 Ex 13-18 were generated;

FIG. 15B shows co-localisation analysis for the two antibodies p31Ab and m22Ab;

FIG. 15C shows the specificity of p31Ab for the overexpressed polypeptide in mCherry FMN2 Ex 13-18;

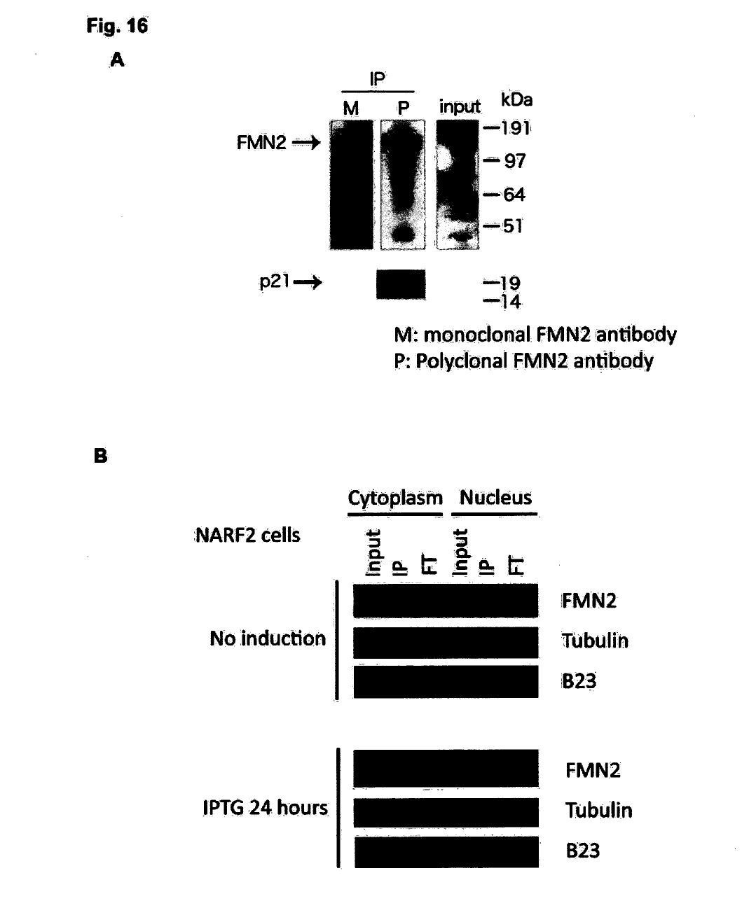

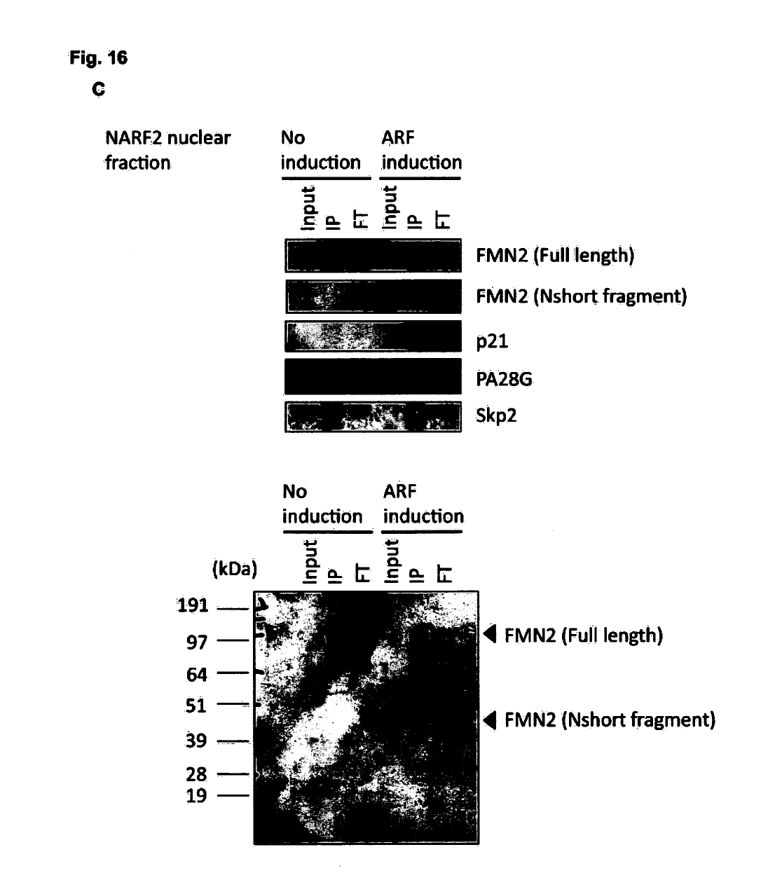

FIG. 16A shows the results of immunoprecipitation (IP) analysis using the FMN2 antibodies p31Ab and m22Ab;

FIGS. 16B and C shows further IP analysis;

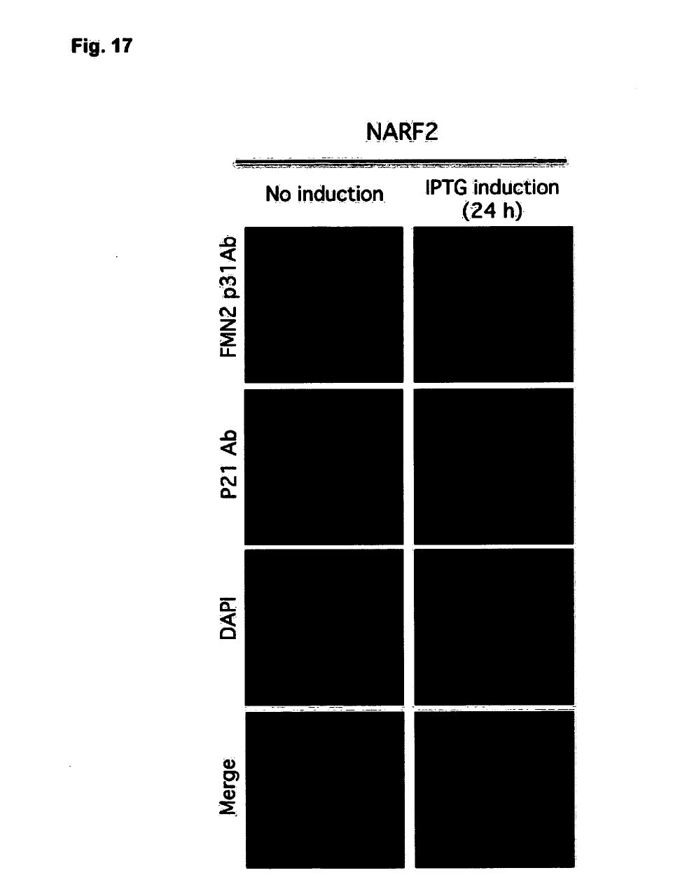

FIG. 17 shows the co-localisation of FMN2 and p21 (CIP1);

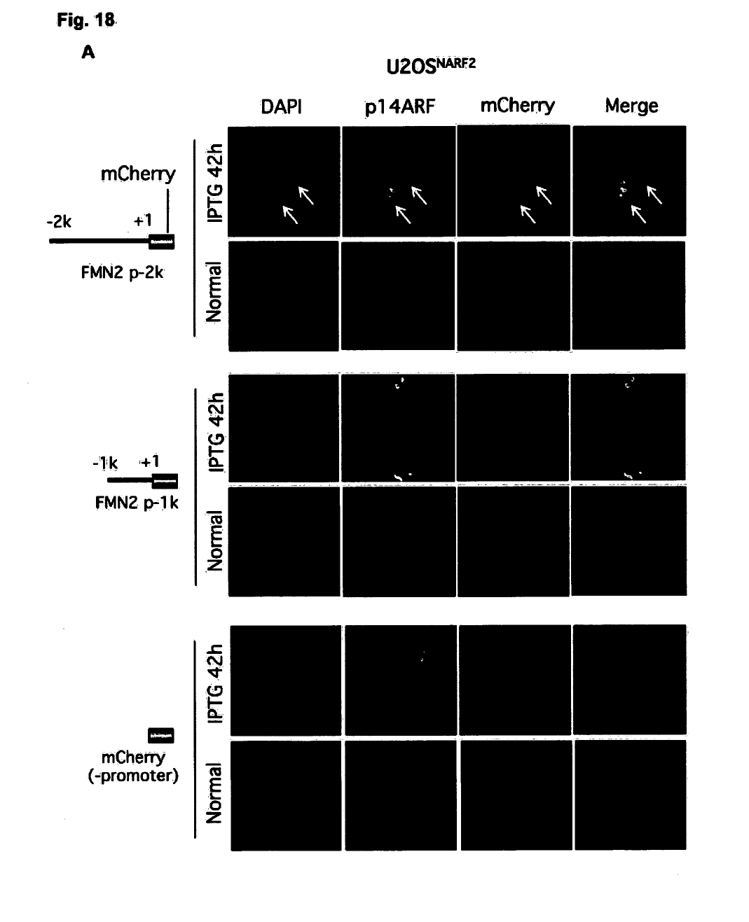

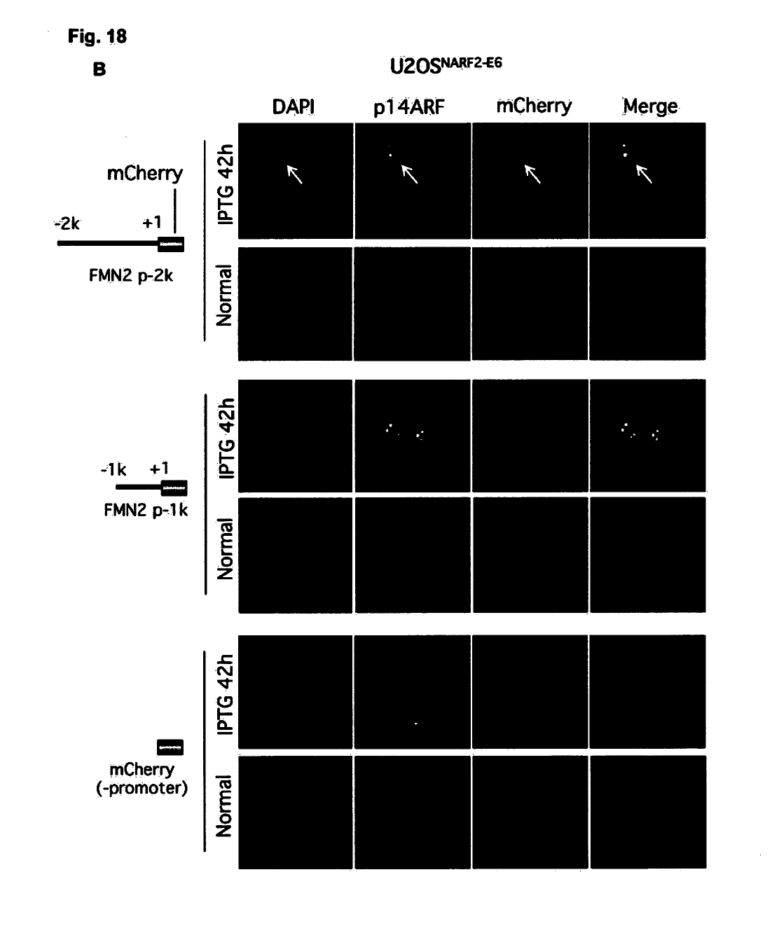

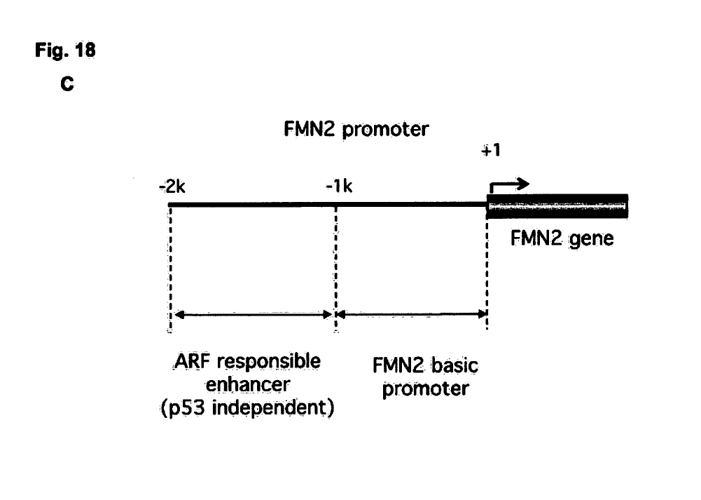

FIGS. 18A and 18B shows the results of studies into detecting the promoter for FMN2;

FIG. 18C shows a schematic representation of the promoter and ARF responsive enhancer element of FMN2; and

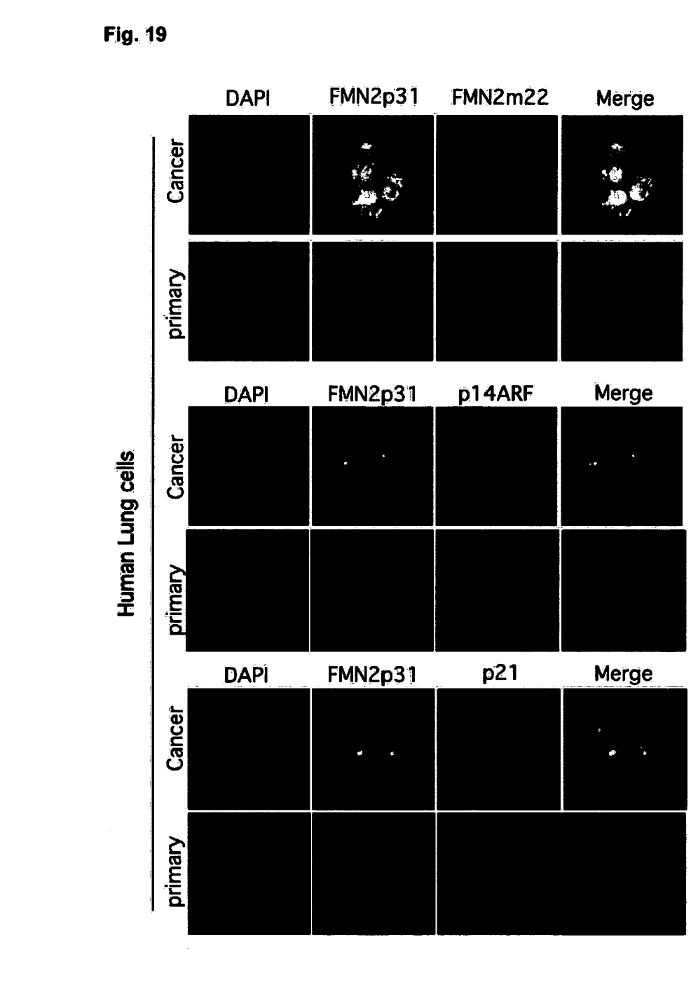

FIG. 19 shows the results of FMN2 expression analysis between lung primary and cancer cells; and

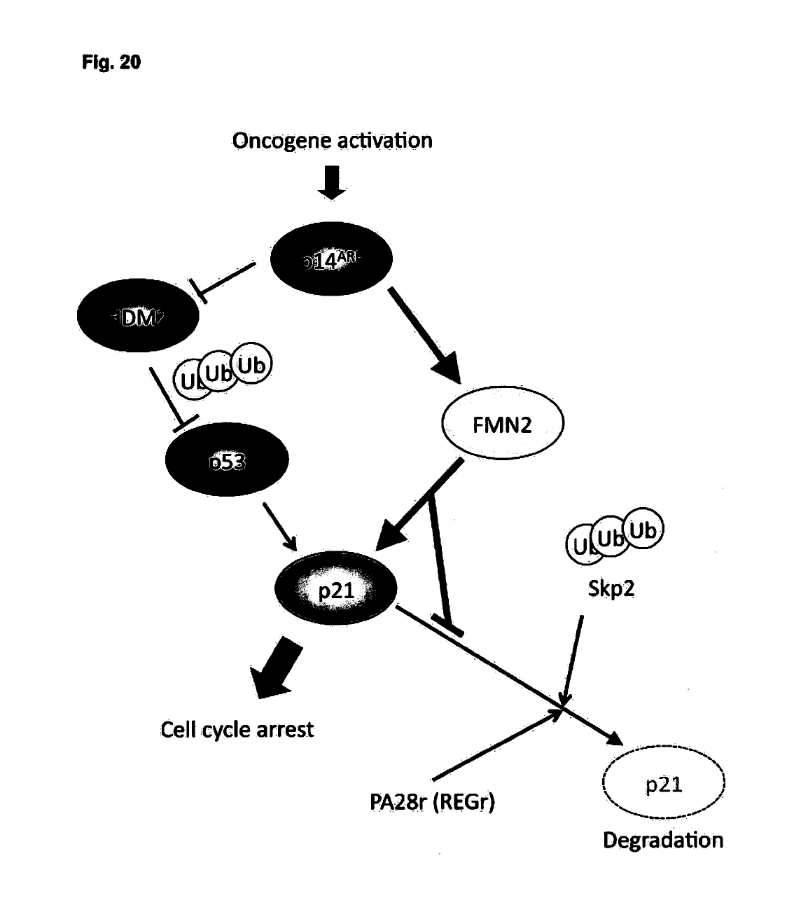

FIG. 20 shows a model of the p14 ARF-FMN2 pathway.

### MATERIALS AND METHODS

### Isolation of Stable Isotope-Labelled Nucleolar Proteins

Cells were grown for at least five cell divisions in L-arginine-, L-arginine ¹³C₆ ¹⁴N₄-, or L-arginine ¹³C₆ ¹⁵N₄-labelling media before ARF induction. For induction of exogenous p14ARF, IPTG was added at a final concentration of 1 mM to all cells and incubated for 4, 8, 16, and 24 hours, respectively. The experiment was repeated with to give a total of five time points with untreated Arg0 cells as a common zero time point. Nucleoli were isolated from NARF2 and E6 as previously described (http://www.lamondlab.com/f5nucleolarprotocol.htm). Isolated nucleolar proteins were separated on NuPAGE 4-12% Bis-Tris gel and excised into 12 slices. Peptides resulting from in-gel digestion were extracted from the gel pieces, desalted and concentrated on reverse-phase C18 tips, and eluted into 96-well plates for automated mass spectrometry analysis.

### Mass Spectrometry and Data Analysis

Mass spectrometric analysis was performed by liquid chromatography (Agilent HP1100) combined with tandem mass spectrometry (LC MS/MS) using a LTQ Obitrap (ABI). For the LTQ Obitrap, precursor ion spectra (m/z 350-1,500) and product ion spectra (m/z 70-1,500) of the four most intense ions were collected for 1 s. The LTQ-FT-ICR instrument was operated in the data-dependent mode to acquire high-resolution precursor ion spectra (m/z 300-1,500, R ¼ 25,000 and ion accumulation to a target value of 10,000,000) in the ICR cell. The three most intense ions were sequentially isolated for accurate mass measurements by selected ion monitoring (SIM) scans (10 Da mass window, R ¼ 50,000, and a target accumulation value of 50,000). The ions were simultaneously fragmented in the linear ion trap with a normalized collision energy setting of 27% and a target value of 2,000.

Stringent criteria were required for protein identification in the International Protein Index database using the Mascot program (Matrix Science) and LTQ-FT-ICR data: at least two matching peptides per protein, a mass accuracy within 3 p.p.m. (average absolute peptide mass accuracy was 0.7 p.p.m.), a Mascot score for individual peptides of better than 20, and a delta score of better than 5. Experiments with a reversed database26 indicated that, under these conditions, proteins with two matching peptides were identified with a false positive rate of less than 0.1 per cent. Protein ratios were calculated for each arginine-containing, peptide as the peak area ratio of Arg6/Arg0 and Arg10/Arg0 of each single scan mass spectrum. The peptide ratios were averaged for all arginine-containing peptides sequenced for each protein and normalized to zero (x−1). Normalized inverted ratios were calculated for ratios smaller than one [1−(1/x)]. MS-Quant (http://msquant.sourceforge.net/), an in-house developed software program was used to evaluate the certainty in peptide identification and in peptide abundance ratio.

### Live Cell Imaging

GFP-ARF cells were cultured in Willco thin glass-bottomed microwell dishes (Intracel), mounted on a Deltavision Spectris microscope (Applied Precision) fitted in a transparent environmental chamber (Solent Scientific). Cells were imaged 60×(NA 1.4) Plan Apochromat objective. Twelve optical sections separated by 0.5 mm were recorded for each field and each exposure lasted for 0.05 s. After recording the first time points, doxycycline was added at a final concentration of 5 μg ml⁻¹ and cells were imaged for 2-3 h (SoftWoRx image processing software, Applied Precision). Nucleoli or nuclei (see FIG. 6) were outlined manually and five nucleoli/nuclei were measured from two independent experiments.

siRNA

siRNA duplex oligonucleotides were synthesised by MWG and transfected using Interferin (Polyplus) as per manufacturers instruction. In brief, cells were plated the day before transfection at the concentration of 2×10⁵ cells per well in 6 well plates. The following day, cells were transfected with the final concentration of 5 nM of siRNA oligonucleotides in fresh media, final volume of 2.2 ml. Cells were incubated for additional 48 hours prior to harvesting. IPTG was added for 24 hours unless otherwise stated. siRNA sequences were described here (Control: CAGUCGCGUUUGCGACUGG, FMN2-GUAUACCAGGUCUCCUCAA). MG132 was purchased from Merck Chemicals and used at the final concentration of 50 μM.

### Cells

NARF2 and NARF2-E6 cell lines were provided by Dr. Gordon Peters (Cancer Research UK London Research Institute) and have been described previously (Stott et al, 1998; Brookes et al, 2002; Rocha et al, 2003). NARF2 cells, a derivative of the human osteosarcoma U2OS cells containing an isopropyl β-D-thiogalactopyranoside (IPTG)-inducible p14^{ARF} gene, have been described previously (Stott et al., 1998). The NARF2-E6 cells are a derivative of NARF2 cells but contain, in addition, constitutively expressed human papillomavirus (HPV) E6 protein.

### RNA Preparation and Labelling.

Total NARF2 cell RNA were isolated using the RNeasy mini kit (QUIAGEN), with DNase I treatment, according to manufacturer's instruction. Total RNAs were labelled with One-Color Microarray-Based Gene Expression system (Agilent Technologies).

### Microarray Experiment and Analysis.

All microarray experiments have proceeded in EMBL's Genomics Core Facility in Heidelberg, Germany. Labeled cDNA quality were analysed by NanoDrop ND-1000 UV-VIS Spectrophotometer. Labeled cDNA were hybridized on Whole Human Genome Oligo DNA microarray (Human WG 4×44 k: Agilent Technologies). Scanned data were analysed by GeneSpring GX software according to manufacture's instruction (Agilent Technology).

### Results

We have analysed the nucleolar protein dynamics of the ARF tumour suppressor pathway using mass-spectrometry-based organellar proteomics and stable isotope labelling, i.e., SILAC (FIG. 5a) (Ref.4-6). We performed a quantitative analysis of the nucleolar proteome from a human ARF inducible model cell line, called NARF2, and comparing with another ARF inducible and p53 negative cell line, called E6 (Ref.1-3). NARF2 is a stable cell line established from U2OS human osteosarcoma cells. Endogenous ARF expression is prevented in U2OS cells by hyper methylation of the ARF gene promoter region. However, these cells also contain an exogenous, inducible copy of the wild type ARF gene whose expression can be induced in NARF2 cells by addition of IPTG. The E6 cell line is established from NARF2 and expresses human papillomavirus (HPV) E6 protein that inactivates p53. We detected peptides from ARF protein (FIG. 5b) and compared that dynamic time course change in NARF2 and E6 cell line (FIG. 5c). The result showed that ARF protein level was dramatically increased in both cell lines during IPTG induction as we expected. Next, we compared mass spectrometry data with signal intensity from fluorescence microscope Live-cell image (FIG. 6). The datasets suggested that fold change data of the ARF was matched with the signal intensity from our microscope (FIG. 6a). We chose some other proteins to compare with our masspec data, and their behaviours were matched with the fold change data that we detected (FIG. 6b).

The data document time course changes in the levels of thousands of nucleolar proteins during ARF induction (FIG. 7a&b). For example, Formin-2 (FMN2) (Ref. 7&8), which has a role in cytokinesis and from our data in cancer, is dramatically increased during induction of ARF both in p53 positive and negative cell lines (FIG. 7b&c). This result suggests that Formin-2 could be a novel partner of the ARF protein or may mediate ARF-dependent downstream mechanisms.

We performed that immunocytochemistry to confirm the expression of FMN2 with or without IPTG induction in these cell lines (FIG. 8). Although FMN2 was expressed at low levels without IPTG in NARF2 and E6 cell lines, the expression level was increased 24 hours after IPTG treatment. We didn't detect such a change in U2OS cells showing that FMN2 induction is not a result of IPTG treatment per se. We also checked the FMN2 expression level in HeLa cells because this cell line has endogenous ARF expression. We detected more FMN2 expression than in U2OS cells which do not express endogenous ARF. These data indicate a positive correlation between ARF and FMN2 expression.

We also performed microarray analysis to assay the RNA levels of these genes (FIG. 9). The FMN2 RNA level showed a high ratio of 10 fold or more increase after induction of exogenous ARF. This result emphasizes that FMN2 mRNA expression was controlled by ARF and thus ARF is affecting the expression of the FMN2 promoter, either directly, or indirectly.

To analyse the function of the FMN2 protein in cells, we designed five siRNAs to suppress FMN2 expression. One of them was successful in suppressing FMN2 expression (FIG. 10). We didn't see any effect on p53 or Hdm2, which is an E3 ubiquitin ligase, but the cyclin dependent kinase inhibitor p21 was severely reduced. (FIG. 10a). Other experiments showed that FMN2 knock-down does not affect puma levels but does reduce p21. In contrast, siRNA knock-down of DNA-PK causes a decrease in the levels of both puma and p21 (FIG. 10b). Therefore, FMN2 shows specificity, and either stabilizes the levels of the p21 protein and/or accelerates p21 expression during oncogene activation. These findings are reproducible and show that FMN2 expression either stabilizes p21, or enhances its expression or both (FIG. 10c).

### Further Materials and Methods

siRNA experiments. siRNA duplex oligo ribonucleotides were synthesised by MWG and transfected using Interferin (Polyplus) as per manufacturers instruction. In brief, cells were plated the day before transfection at the concentration of 2×10⁵ cells per well in 6 well plates. The following day, cells were transfected with the final concentration of 5 nM of siRNA oligoribonucleotides in fresh media, final volume of 2.2 ml. Cells were incubated for a further 48 hours prior to harvesting. IPTG was added for 24 hours unless otherwise stated. siRNA sequences are described here (Control: 5′-CAGUCGCGUUUGCGACUGG-3′, FMN2-5′-GUAUACCAGGUCUCCUCAA-3′, PA28γ-5′-GAAUCAAUAUGUCACUCUA-3′, Skp2-5′-ACUCAAGUCCAGCCAUAAG-3′).).

Antibody production. FMN2 mouse monoclonal and rabbit polyclonal antibodies were produced by Dundee Cell Products (Dundee, UK). The oligopeptides (CTEHVRAPPAPSRSR, MHSIRTVEIKVPEIEEC, KDSQALQTGELDSAHS) for TC supernatant of mouse ascites hybridoma clone were synthesised to establish FMN2-m22Ab (FIG. 15A) and oligopeptides (CRQKKGKSLYKIKPR, CKPRHDSGIKAKISMKT) were synthesised to establish FMN2-p31Ab.

Immunoprecipitation. Immunoprecipitations were carried out as previously described (Trinkle-Mulcahy et al., 2006). Nuclear lysates were prepared from NARF2 stable cell lines. Purified nuclei were resuspended in RIPA buffer to solubilize proteins. FMN2 proteins were immunoprecipitated using an anti-FMN2-m22Ab monoclonal antibody and an anti-FMN2-p31Ab (FIG. 15). Samples were divided in two and for Input samples were isolated from one half of each nuclear lysate.

### Microscopy

All cell images were recorded using the DeltaVision Spectris fluorescence microscope (Applied Precision). Cells were imaged using a 60× (NA 1.4) Plan Apochromat objective. Twelve optical sections separated by 0.5 μm were recorded for each field and each exposure (SoftWoRx image processing software, Applied Precision).

### Results

FIG. 14. Shows the rescue of FMN2 depletion with siRNA for PA28γ and Skp2. Detection of protein levels for endogenous FMN2, Skp2, PA28G, p21, p14ARF and Actin following transfection of NARF2 cells using either siRNAs for FMN2 (see FIG. 15A for location of siRNA target sequence), PA28γ, Skp2 and control (Control) with/without p14ARF induction (+/blank). An equivalent amount of NARF2 extract was loaded for each lane and the proteins separated by SDS PAGE, electroblotted and probed both with a monoclonal anti-PA28γ and polyclonal anti-FMN2, anti-Skp2, anti-p21, anti-p14ARF and with anti-Actin as a loading control. The p21 inactivation observed following FMN2 depletion was partially rescued by co-transfection with both PA28γ siRNA/Skp2 siRNAs. These data suggesting that p21 inactivation by FMN2 depletion is caused by both a ubiquitin dependent degradation pathway (Skp2) and ubiquitin independent pathway (PA28γ) (a model is shown in FIG. 20).

Establishment of FMN2 specific antibodies. a. Mouse monoclonal anti-FMN2 antibody (m22Ab) and Rabbit polyclonal anti-FMN2 antibody (p31Ab) were established by injecting synthesised FMN2 oligopeptides (see FIG. 15A). To confirm the antibody specificity, FMN2 partial cDNA sequence was fused with mCherry red fluorescence protein cDNA (mCherry-FMN2Ex6-12 & mCherry-FMN2Ex13-18). FIG. 15A also shows the FMN2 protein structure and indicates the positions for siRNA used for FMN2 knock-down, peptides sequences used as antigens to raise anti-FMN2 antibodies, and motifs. The FH2 domain is almost entirely α helical and can be subdivided into five subdomains with somewhat arbitrary boundaries. These include an N-terminal “lasso”, a “linker” segment, a globular “knob” subdomain, a coiled-coil region, a carboxy-terminal “post2 subdomain The dimer formation is mediated by a unique interactions of “lasso” with “post” of the partner FH2, exhibiting a closed ring structure.

It has been proposed that the DEP domain could play a selective role in targeting DEP domains-containing proteins to specific subcellular membranous sites, perhaps even to specific G protein-coupled signaling pathways.

FIG. 15B shows co-localisation analysis for FMN2 m22Ab-p31Ab. NARF2 cells were fixed and stained with mouse monoclonal anti-FMN2 antibody (m22Ab) and rabbit polyclonal anti-FMN2 antibody (p31Ab) following p14ARF induction.

FIG. 15C shows the results of Expression of mCherry-FMN2 partial plasmids. HeLa cells were fixed and stained using polyclonal anti-FMN2 antibody (p31Ab) after transfection with pmCherry-FMN2Ex6-12 and pmCHerry-FMN2Ex13-18. Scale bar is 10 μm. The arrows indicate transfected cells. As can be seen transient expression of pmCHerry-FMN2Ex13-18 which includes the antigen sequence recognised by FMN2-p31Ab, was strongly stained by FMN2-p31Ab. This same FMN2-p31Ab antibody did not stain cells after transient expression of pmCHerry-FMN2Ex6-12, which doesn't include the peptide sequences used as antigens to raise anti-FMN2 antibodies (Arrow).

FIG. 16. shows the results of Immunoprecipitation (IP) analysis using FMN2 specific antibodies. Immunoprecipitations were performed as previously shown (Trinkle-Mulcahy et al., 2006). NARF2 cell nuclear lysates were isolated after p14ARF induction and equal amount of lysates were mixed with either the FMN2 polyclonal antibody (FMN2p31Ab) or with monoclonal antibody (FMN2m22Ab). Fractionation was also confirmed by checking the B23 protein as a nuclear marker and tubulin as a cytoplasmic marker (FIG. 16B). An equivalent amount of IP extract was loaded for each lane and the proteins separated by SDS PAGE, electroblotted and probed with anti-FMN2 antibody e.g. IP with a monoclonal anti-FMN2 and blotting with polyclonal anti-FMN2 (lane M) (See FIG. 15A). As can be seen, clear isolation of FMN2 was shown after IP compared with Input lane (lysate without antibody). p21 was also detected in the FMN2 IP.

NARF2 cell nuclear lysates are isolated with/without p14ARF induction and equal amount of lysates were mixed with FMN2 polyclonal antibody (FMN2 p31Ab). An equivalent amount of the total nuclear lysate (Input), precipitated sample (IP) and flow-through samples (FT) was loaded for each lane and the proteins separated by SDS PAGE, electroblotted and probed with antibodies including anti-FMN2 m22Ab, anti-PA28γ, anti-Skp2 and anti-B23 as a loading control for Input and FT (see FIG. 16C).

NARF2 cells were fixed and stained with rabbit anti-FMN2 p31Ab and anti-p21 antibody with/without p14ARF induction. FIG. 17 shows that majority of FMN2 and p21 signals were both co-localised in the nuclei. This result is consistent with the IP result presented in FIG. 15.

Two FMN2 promoter deletion plasmids upstream of first ATG (+1) were constructed (left panel FIG. 18A). NARF2 cells were fixed and stained with anti-p14ARF antibody with/without p14ARF induction following transfection with plasmids mCherry-FMN2p-2k, mCherry-FMN2p-1k or mCherry without promoter. As can be seen mCherry-FMN2p-2k which has approximately 2000 bases of upstream sequence of the FMN2 gene was upregulated in p14ARF induced cells. However, mCherry-FMN2p-1k which has only 1000 bases of upstream sequence from the FMN2 gene did not show upregulation in response to p14ARF induction (Arrow) (See FIG. 18A right-hand panel). The same experiment was carried out with NARF2-E6 (p53 negative cell) cells (see FIG. 18B). This shows the same result as seen with NARF2 cells. These results indicate that the FMN2 promoter/regulatory region upstream of the FMN2 gene (approximately −2000 bases) includes one or more elements which confer induction by ARF that is p53 independent.

Finally, FIG. 18C shows a Summary of the FMN2 promoter analysis. The FMN2 promoter region was also characterised and the ARF inducible element was determined (Chromosome 1,+strand approximately from 238319604 to 238321603, Genome browser).

Human lung primary fibroblast cells (ATCC-CCL-211) and Human lung Adenocarcinoma cells (ATCC-CRL-5868) were fixed and stained with anti-FMN2-p31Ab, anti-FMN2-m22Ab, anti-p21 and anti-p14ARF antibodies. FIG. 19 shows that the FMN2 gene was up regulated in human lung cancer cells, as compared with human lung primary cells, as we observed in tissue culture cells such as U205, HeLa, NARF2, and NARF2-E6 cell lines, i.e. enhanced FMN2 expression after oncogene activation (See also FIG. 8). These data show that elevated FMN2 expression may occur in multiple forms of cancer as well as in model tissue culture systems.

FIG. 20 Model of p14ARF-FMN2 pathway. We found a novel tumour suppressor pathway that is p53 independent. p14ARF induced by oncogene activation up regulates the FMN2 gene independent of p53. FMN2, either directly or indirectly, inhibits p21 degradation including both ubiquitin-dependent and independent pathways.

### REFERENCES

1. Rocha S. and Perkins N. D., ARF the integrator: linking NF-kappaB, p53 and checkpoint kinases. Cell Cycle, 6, 756-9 (2005).

2. Rocha S., Garrett M. D., Campbell K. J., Schumm K., Perkins N. D., Regulation of NF-kappaB and p53 through activation of ATR and Chk1 by the ARF tumour suppressor. EMBO J., 24(6), 1157-69 (2005).

3. Rocha S., Campbell K. J., Perkins N. D., p53- and Mdm2-independent repression of NF-kappa B transactivation by the ARF tumor suppressor. Mol Cell, 1, 15-25 (2003).

4. Andersen, J. S. et al., Directed proteomic analysis of the human nucleolus. Curr. Biol., 12, 1-11 (2002).

5. Andersen, J. S. et al., Nucleolar proteome dynamics. Nature, 433, 77-83 (2005).

6. Lam, Y. W., Lamond, A. I., Mann, M. J., Andersen, S. Analysis of nucleolar protein dynamics reveals the nuclear degradation of ribosomal proteins. Curr. Biol., 17, 749760 (2007).

7. Katoh M., Katoh M., Characterization of FMN2 gene at human chromosome 1q43. Int. J. Mol. Med., 3, 469-74 (2004).

8. Leader B. et al., Formin-2, polyploidy, hypofertility and positioning of the meiotic spindle in mouse oocytes. Nat. Cell. Biol. 12, 921-8 (2002).

9. Trinkle-Mulcahy, L., Andersen, J., Lam, Y. W., Moorhead, G., Mann, M., and Lamond, A. I. (2006). Repo-Man recruits PP1 gamma to chromatin and is essential for cell viability. J Cell Biol 172, 679-692.

10. Sambrook J. et al., Molecular Cloning (Third Edition), CHS press, (2001).

11. Gerd Gellissen (edt.), Production of Recombinant proteins, John Wiley & Sons (2005).

12. Gregory J. Hannon (edt.), RNAi: a guide to gene silencing, Cold Spring Harbor Laboratory Press (2003).

What is claimed is:

1. A method of diagnosing a cell proliferation and/or differentiation disorder, or a susceptibility to developing such a disorder, said method comprising the step of detecting modulation of Formin2 (FMN2) gene and/or protein expression, wherein modulation of FMN2 gene and/or protein expression is indicative of the cell proliferation and/or differentiation disorder.

2. The method according to claim 1 wherein the modulation is an increase in the level of FMN2 gene and/or protein expression.

3. The method according to claim 1 wherein the cell proliferation and/or differentiation disorder is a cancer, such as a carcinoma, sarcoma or lymphoma, leukaemia or an autoimmune and/or inflammatory disorder such as psoriasis, allergies and the like, disorders of meiosis, for example disorders involving the proliferation of germ cells which may result in infertility or diseases and/or conditions such as hepatitis, liver cirrhosis, renal failure, acute pneumonia, stomach ulcer, cardiac infarction, muscular dystrophy and cerebral infarction.

4. The method according to claim 1 wherein the modulation is a decrease in FMN2 gene and/or protein expression may indicate the presence of, or a susceptibility to, developing the cell proliferation and/or differentiation disorders.

5. The method according to claim 4 wherein the cell proliferation and/or differentiation disorder is characterised by aberrant apoptotic events and may be a neurodegenerative disease, such as an acute cellular degenerative disease and/or conditions such as ischemic events (for example, stroke, myocardial infarction and the like).

6. An isolated cDNA sequence comprising one or more of the sequences shown in FIG. 2 or 3 or an isoform, alternative splice form or post-translationally modified or truncated form thereof.

7. A DNA microarray comprising a cDNA according to claim 6 for use in a method of diagnosing a cell proliferation and/or differentiation disorder, or a susceptibility to developing such a disorder, said method comprising the step of detecting modulation of Formin2 (FMN2) gene and/or protein expression, wherein modulation of FMN2 gene and/or protein expression is indicative of the cell proliferation and/or differentiation disorder.

8. A recombinant FMN2 protein or a fragment thereof expressed from the isolated cDNA according to claim 6.

9. A polyclonal or monoclonal antibody or antibody fragment capable of specifically reacting with the recombinant protein or fragment according to claim 8.

10. The antibody or antibody fragment according to claim 9 bound to a substrate for use in a method of diagnosing a cell proliferation and/or differentiation disorder, or a susceptibility to developing such a disorder, said method comprising the step of detecting modulation of Formin2 (FMN2) gene and/or protein expression, wherein modulation of FMN2 gene and/or protein expression is indicative of the cell proliferation and/or differentiation disorder.

11. The antibody or antibody fragment according to claim 9, conjugated to a detectable moiety, such as a colourometric, fluorescent, chemiluminescent or radio label.

12. A kit comprising one or more oligonucleotides/primers which are capable of specifically hybridising to the FMN2 gene or an FMN2 gene comprising one or more mutations.

13. A compound capable of modulating FMN2 gene and/or protein expression/function, for use in treating cell proliferation and/or differentiation disorders.

14. (canceled)

15. A method of treating a cell proliferation and/or differentiation disorder, said method comprising the step of administering to a subject in need thereof a compound capable of modulating FMN2 gene and/or protein expression/function.

16. A method of identifying or obtaining agents which modulate the expression of FMN2 gene and/or protein, said method comprising the steps of contacting the FMN2 gene or protein with a test agent and detecting any modulation of FMN2 gene and/or protein expression/function.

17. A pharmaceutical composition comprising a compound which is capable of modulating expression or function of FMN2 genes/proteins, in association with a pharmaceutically acceptable excipient, carrier or diluent.

18. A vector comprising an ARF responsive enhancer and a promoter for use in expressing a gene designed to treat a cell proliferation and/or differentiation disorder.

19. The vector according to claim 18 wherein the ARF responsive enhancer and promoter comprises a sequence approximately 2 kb upstream of a FMN2 start codon.

20. The vector according to claim 18 wherein the gene is a cytotoxic gene and the disease to be treated is a cancer.

21. A method of treating a cell proliferation and/or differentiation disorder, comprising administering the vector according to claim 18 to a subject.
